(19) United States
(12) Patent Application Publication
(10) Pub. No.: US 2011/0309541 A1
(43) Pub. Date: Dec. 22, 2011

(54) OPEN-CELL SURFACE FOAM MATERIALS

(75) Inventors: Jordan M. Thompson, Scotts Valley, CA (US); Alexei Goraltchouk, Santa Barbara, CA (US); Loantrang T. Dang, Ventura, CA (US)

(73) Assignee: ALLERGAN, INC., Irvine, CA (US)

(21) Appl. No.: 13/160,325

(22) Filed: Jun. 14, 2011

Related U.S. Application Data
(60) Provisional application No. 61/355,305, filed on Jun. 16, 2010.

(51) Int. Cl. B29C 63/26; A61F 2/12; B29C 67/20

(52) U.S. Cl. 264/46.6; 623/8

(57) ABSTRACT

The present specification discloses open cell surface foam materials, methods of forming such foam materials, biocompatible implantable devices comprising such foam materials, and methods of making such biocompatible implantable devices.

This application claims priority to and the benefit of, U.S. Provisional Patent Application No. 61/355,305, filed on Jun. 16, 2010, and the entire disclosure of which is incorporated herein by this specific reference.

Traditional techniques for manufacturing polymer foams often result in foams having a non-porous or closed-cell surface, colloquially referred to as a “skin.” Foam materials are typically manufactured by mixing uncured polymer material with a foaming agent which brings about the formation of gas bubbles in the material. Once cured, the gas bubbles are reflected in the porous structure of the material, thus creating a foam material comprising an elastomer matrix defining an array of interconnected pores. During such processes, however, the porous structure of the surface material often collapses, forming a solid sheet on the surface of the material. This solid sheet or skin covers the underlying pores, resulting in a foam material having a non-porous or closed-cell surface, or at least a surface of less porosity, or less open-cell surface, than the remainder of the material. See, e.g., FIG. 1A.

However, many applications require foam materials having a porous or open-cell surface. As an example, many medical applications involve the permanent or semi-permanent implantation of objects, made of elastomers or other polymers, within the bodies of an individual, such as, e.g., a breast implant, a pacemaker, an artificial joint. Typically, these implantable devices are introduced into an individual's body during a surgical procedure, which involves the creation of an incision, insertion of the implant, and closing of the open wound. The wound then heals around the implant which may be permanently incorporated into the individual's body.

Implantable medical devices frequently induce a foreign body response that results in the formation of an avascular, fibrous capsule around the implant, which limits the performance of the device. For example, formation of these fibrous capsules can result in capsular contracture, the tightening and hardening of the capsule that surrounding implanted device. Capsular contractions not only distort the aesthetic appearance of the surrounding area where the implant is placed, but also cause pain to the individual. Problems with capsular formation and contracture occur in many types of implantable medical devices, such as, e.g., pacemakers, orthopedic joint prosthetics, dura matter substitutes, implantable cardiac defibrillators, tissue expanders, and tissue implants used for prosthetic, reconstructive, or aesthetic purposes, like breast implants, muscle implants, or implants that reduce or prevent scarring. Correction of capsular contracture may require surgical removal or release of the capsule, or removal and possible replacement of the device itself.

Scar tissue formation in the healing of a wound or surgical incision is also a process involving the formation of fibrous tissue. A visible scar results from this healing process because the fibrous tissue is aligned in one direction. However, it is often aesthetically desirable to prevent scar formation, especially in certain types of plastic surgery.

The biological response to implantable medical devices and wound healing appears dependent on the microarchitecture of the surface of the implants. Implants with smooth surfaces, such as, e.g., foams having a non-porous or closed-cell surface, in particular are most susceptible to capsular formation and contracture.

As such, there is a continuing need for implantable medical devices manufactured in such a way that the formation of fibrous capsules is reduced or prevented. The present application discloses foams with an open-cell surface, methods of making these foams, implantable medical devices comprising such foams, and methods of making such implantable medical devices. The foams with an open-cell surface promote cellular ingrowth in and around an implantable medical device and reduce or prevent a foreign body response, such as, e.g., capsular contracture as well as to reduce or prevent scars resulting from wound healing.

Thus, aspects of the present specification disclose an open-cell surface foam material comprising a three-dimensional non-degradable, biocompatible, elastomer matrix defining an array of interconnected pores.

Other aspects of the present specification disclose a method making an open-cell surface foam material, the method comprising the steps of: a) preparing a foaming solution, the foaming solution comprising an elastomer base and a curing agent; b) adding the foaming solution to a mold assembly, the mold assemble comprising a textured surface component having an interconnected array of void space, wherein the foaming solution is added to the mold assembly in a manner sufficient to allow penetration of the foaming solution into the interconnected array of void space; c) treating the foaming solution to produce a foam material; and d) removing the mold assembly, wherein removal of the textured surface component produces a foam material having an open-cell surface.

Yet other aspects of the present specification disclose an open-cell surface foam material comprising an elastomer matrix defining an array of interconnected pores, wherein the foam material is made by the method comprising the steps of: a) preparing a foaming solution, the foaming solution comprising an elastomer base and a curing agent; b) adding the foaming solution to a mold assembly, the mold assemble comprising a textured surface component having an interconnected array of void space, wherein the foaming solution is added to the mold assembly in a manner sufficient to allow penetration of the foaming solution into the interconnected array of void space; c) treating the foaming solution to produce a foam material; and d) removing the mold assembly, wherein removal of the textured surface component produces a foam material having an open-cell surface.

Still other aspects of the present specification disclose a biocompatible implantable device comprising a layer of open-cell surface foam material disclosed in the present specification. The open-cell surface foam can be made by the methods disclosed in the present specification.

Further aspects of the present specification disclose a method of making biocompatible implantable device comprising the steps of: a) preparing the surface of a device to receive an open-cell surface foam material; b) attaching an open-cell surface foam material to the prepared surface of the device. The open-cell surface foam material can be made by the method disclosed in the present specification.

### BRIEF DESCRIPTION OF THE DRAWINGS

FIG. 1A is a simplified schematic diagram showing a PRIOR ART method and system for making a foam material.

FIG. 1B is a simplified schematic diagram showing a method and apparatus in accordance with an embodiment of the present invention.

FIG. 1C is a simplified schematic diagram showing a method and apparatus in accordance with another embodiment of the present invention.

FIGS. 2A and 2B illustrate front and side view of a representative biocompatible implantable device covered with a porous material in made in accordance with a method of the present invention.

FIGS. 2C and 2D illustrate simplified cross-sectional views of the device shown in FIGS. 2A and 2B.

FIGS. 3A-3D are front, side, back and cross-sectional views, respectively, of a material shell made with a method of the invention.

FIGS. 4A-4D are front, side, back and cross-sectional views, respectively, of biocompatible implantable device covered with a porous material of the present invention.

### DETAILED DESCRIPTION

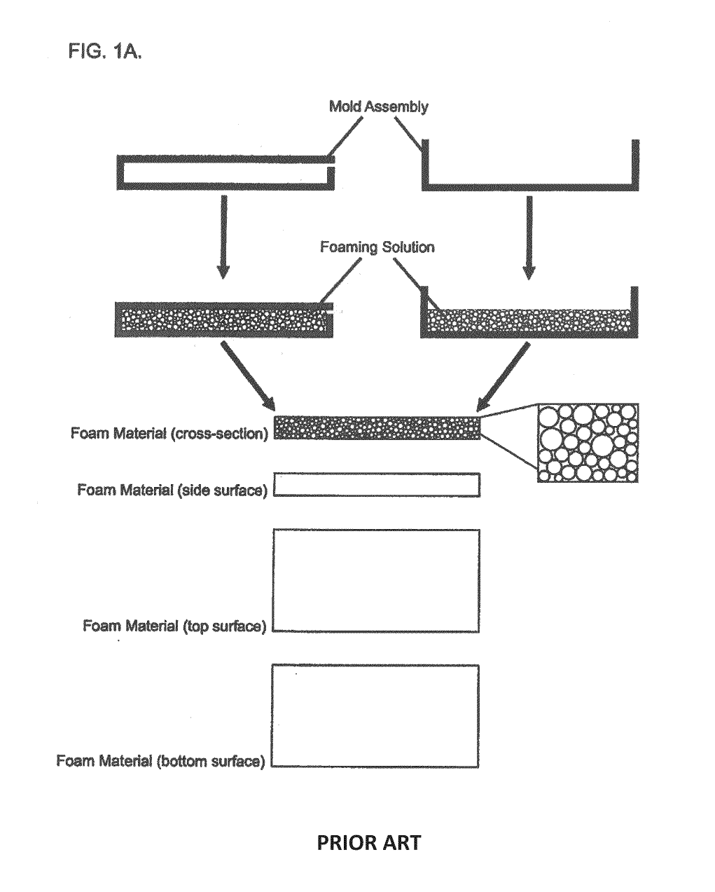

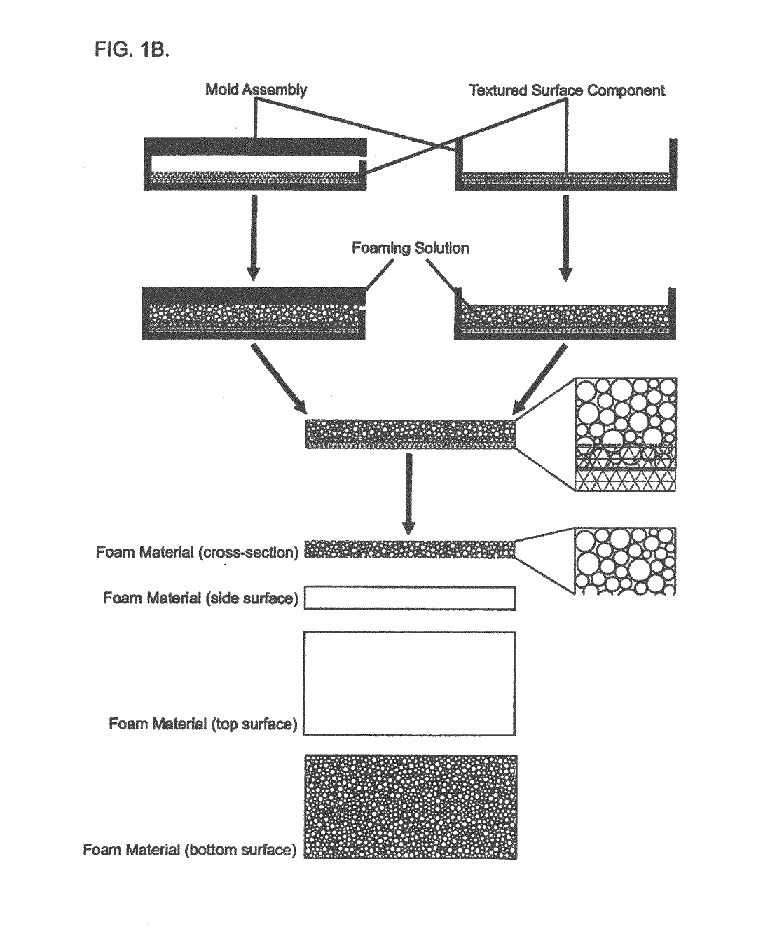

FIG. 1A illustrates a conventional method of making a foam in which a foaming solution is added to a mold assembly by injection via an injection port (assembly on left) or by simply pouring the solution into a mold assembly (assembly on the right). Whether by contact with a surface of the mold assembly or by exposure to air, a “skin develops all surfaces of the foam material as it cures. As such, a mold assembly as illustrated in FIG. 1A produces a foam material where all surfaces are closed cell surfaces. FIG. 1B illustrates the use of a mold assembly comprising a component having a textured surface. In this case, after adding a foaming solution to a mold assembly, the solution penetrates or integrates into the textured surface component. After the foaming solution cures, the textured surface component is removed producing an open cell surface where the cured foam material was in contact with the textured surface component. As such, a mold assembly as illustrated in FIG. 1B produces a foam material having an open cell surface. FIG. 1C illustrates the use of a mold assembly comprising a plurality of components having a textured surface. As in FIG. 1B, removal of the textured surface components produces an open cell surface where the cured foam material was in contact with the textured surface components. The only difference is that multiple open cell surfaces can be produced on the foam material. As such, a mold assembly as illustrated in FIG. 1C produces a foam material having a plurality of open cell surfaces.

In one aspect of the invention, an implantable device, for example, a breast implant shell is provided comprising a fillable envelope including a porous material for example, an open-cell foam element, defining an anterior surface of the envelope. The open-cell foam element is produced by preparing a foaming solution comprising an elastomer base and a curing agent and adding the foaming solution to a mold assembly having a textured surface component. In certain advantageous embodiments of the invention, the mold assembly has a high surface porosity, for example, a surface porosity of at least about 40% or greater. The textured surface is defined by an interconnected array of void space and the foaming solution is added to the mold assembly in a manner sufficient to allow penetration of the foaming solution into the interconnected array of void space. The foaming solution is treated or cured to produce an elastomeric foam material on the textured surface component. Next, the elastomeric foam material is removed from the textured surface component thereby obtaining a foam material having an open-cell surface.

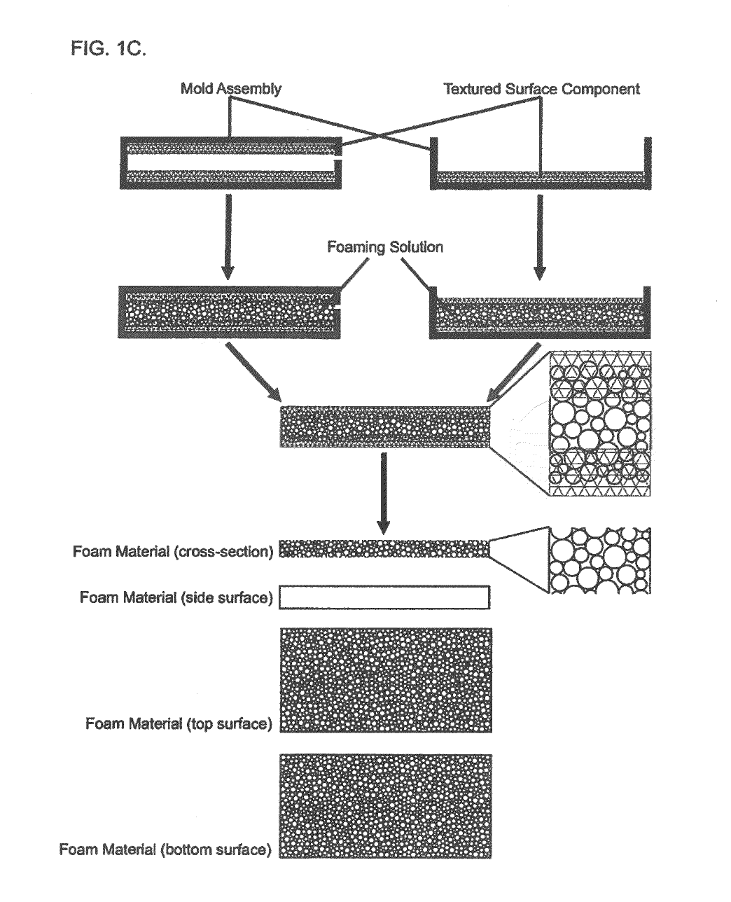

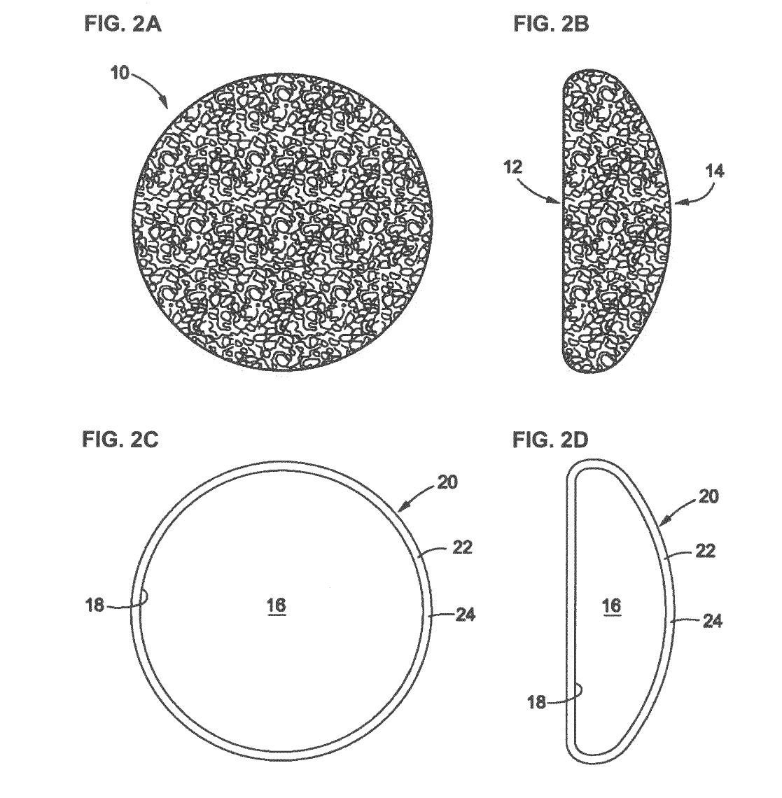

FIGS. 2A-2D illustrate a representative biocompatible implantable device 10, for example, a fillable breast implant shell, covered with a porous material, for example, an open-cell foam material, of the present invention. FIG. 2A is a top view of an implantable device 10 covered with the porous material. FIG. 2B is a side view of an implantable device 10 covered with the porous material to show a posterior or bottom 12 of the implantable device 10 and a convex anterior or top 14 of the implantable device 10. FIGS. 2C and 2D illustrate cross-sectional views of a biocompatible implantable device 10 to show a fillable cavity or interior 16, elastomeric, solid interior surface 18, and elastomeric porous outer surface 20 defined by an open cell foam material of the invention, the solid interior surface 18 and porous outer surface forming surfaces of the fillable shell 22 having a textured, open-cell surface 24 which may promote healthy tissue ingrowth and reduce the occurrence of capsular contracture.

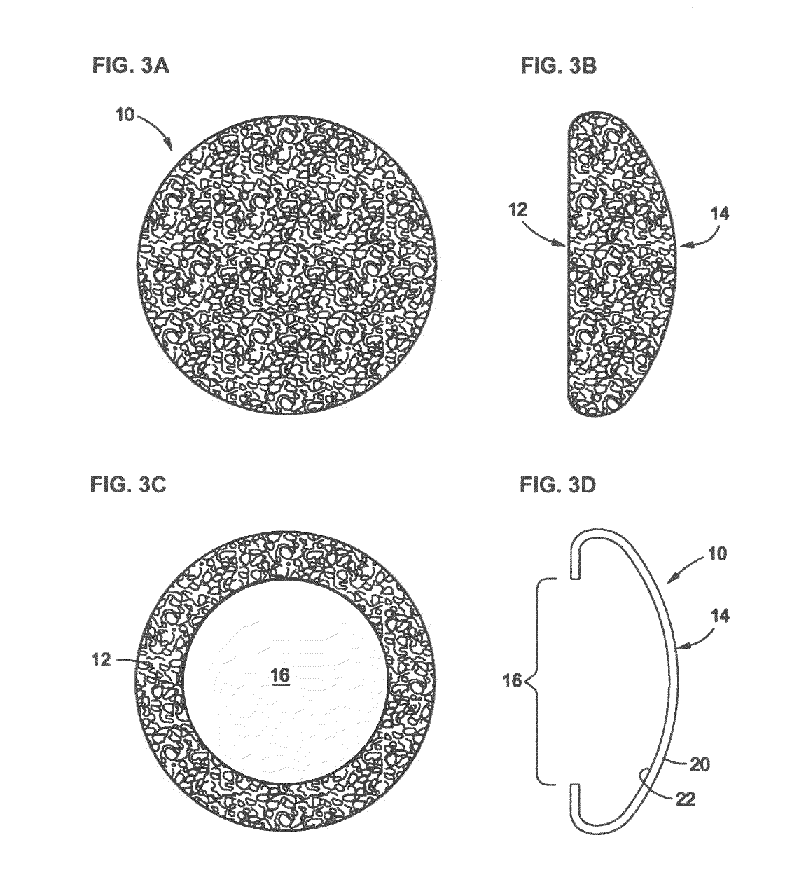

FIGS. 3A-3D illustrate a representative porous material shell 10 of the present specification. FIG. 3A is a top view of a material shell 10. FIG. 2B is a side view of a material shell 10 to show a bottom 12 of the material shell 10 and a top 14 of the material shell 10. FIG. 3C is a bottom view of a material shell 10 to show a hole 16 from which a biocompatible implantable device may be subsequently inserted through. FIG. 3D illustrate the cross-sectional view of the material shell 10 to show the hole 16, an internal surface 20 of the material shell 10 and an external surface 22 of the material shell 10.

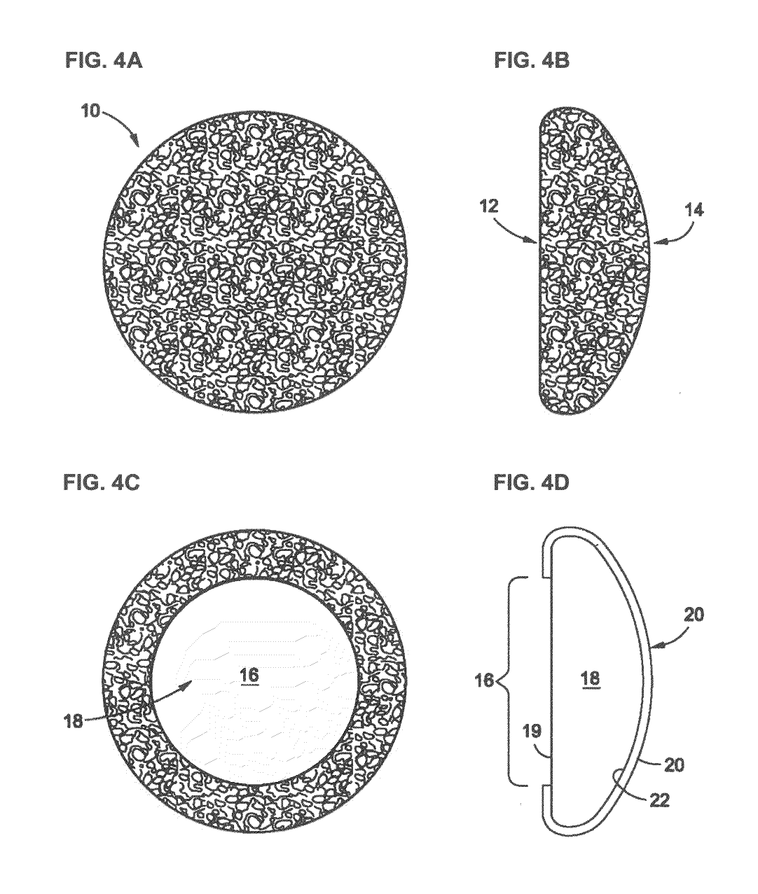

FIGS. 4A-4D illustrate a representative biocompatible implantable device covered with a porous material 10 of the present specification. FIG. 4A is a top view of an implantable device covered with a porous material 10. FIG. 4B is a side view of an implantable device covered with a porous material 10 to show a bottom 12 of the implantable device 10 and a top 14 of the implantable device 10. FIG. 4C is a bottom view of a biocompatible implantable device covered with a porous material 10 to show a hole 16 and an implantable device 18. FIG. 4D illustrates the cross-sectional view of the biocompatible implantable device covered with a porous material 10 to show an implantable device 18, a porous material layer 20 including an internal surface 22 and an external surface 24, where the internal surface 22 is attached to implantable device surface 19. Due to the presence of the porous material on the device surface of the biocompatible implantable device there will be a reduction or prevention of the formation of fibrous capsules that can result in capsular contracture or scarring.

As explained above, traditional techniques for manufacturing polymer foams often result in foams having a non-porous surface, or a surface with less porosity than the remainder of the material, which is not desirable in many applications. In particular, using traditional foaming techniques, a non- or less-porous skin may form on some or all of the foam's surfaces. The formation of such a skin may occur as the foaming solution, which will form the finished foam, makes contact with a surface of a component from a mold assembly, or is exposed to the atmosphere. This contact or exposure causes the foaming solution to collapse at such contacts or exposure, which upon curing of the foaming solution forms a smooth or non-porous surface of the foam material, i.e., a skin.

Traditional manufacturing techniques address the creation of skins by cutting off such skins after the foaming process is complete in order to create a foam material which has an open-cell surface throughout the material. For example, in some processes, foams are made into thick sheets, fixed to a revolving mandrel, and sliced with a blade in a peeling fashion in order to produce sheets of foam material having an open cell surface. Such methods, however, are able to produce foams in only a limited number of geometries, and are also expensive to implement, requiring the use of additional manufacturing steps and cutting machinery. Such techniques are generally limited to creating foam material I the shape of a sheet, and cannot be applied to the creation of objects comprising a open cell surface foam material having more complicated surfaces, such as, e.g. custom molded shapes. In fact, the creation of objects comprising an open cell surface foam material with more complicated geometries may be particularly difficult, as such objects often have many surfaces where skins may form.

Accordingly, the present specification discloses, in part, an open-cell surface foam material. As used herein, the term “open cell” is synonymous with “surface porosity” and refers to openings on the surface of a foam material that connect with the pores present in the foam material. The disclosed open-cell surface foam material has high surface porosity (many open cells on the surface) and interconnected pores that favors tissue growth into the open-cell surface foam material, such as, e.g., by facilitating cell migration, cell proliferation, cell differentiation, nutrient exchange, and/or waste removal. The high surface porosity and interconnected void space structure encourages cell infiltration and growth therein, which disrupt the planar arrangement of capsule formation. High surface porosity and interconnected of the pores is achieved without sacrificing mechanical strength of the foam material, that is, the material's hardness, tensile strength, elongation, tear strength, abrasion and resistance, are preserved. As such, the open-cell surface foam material, its application in creating biocompatible implantable devices, and other aspects disclosed herein are useful in preventing capsular contraction, and in reducing or preventing scar formation.

Even further, it is often important to anchor a biocompatible implantable device to the surrounding tissue in order to prevent slippage or unwanted movement. For example, it is important to anchor securely facial and breast implants into position because of their prominent locations. As such, the open-cell surface foam material, its application in creating biocompatible implantable devices, and other aspects disclosed herein are useful in anchoring biocompatible implantable devices.

An open-cell surface foam material disclosed in the present specification can be implanted into the soft tissue of an animal during the normal operation of the device. Such foam material may be completely implanted into the soft tissue of an animal body (i.e., the entire material is within the body), or the device may be partially implanted into an animal body (i.e., only part of the material is implanted within an animal body, the remainder of the material being located outside of the animal body). An open-cell surface foam material disclosed in the present specification can also be affixed to soft tissue of an animal, typically affixed to the skin of an animal body. For example, a strip of open-cell surface foam material can be placed subcutaneously underneath a healing wound or incision to prevent the fibrous tissue from aligning and thereby reducing or preventing scar formation.

The present specification discloses, in part, an open-cell surface foam material having a surface porosity sufficient to allow tissue growth into the foam material. As such, the surface porosity should support aspects of tissue growth such as, e.g., cell migration, cell proliferation, cell differentiation, nutrient exchange, and/or waste removal. As used herein, the term “surface porosity” refers to the amount of pore openings present on the surface of a foam material. As such, the total surface of a foam material disclosed in the present specification is based upon the surface comprising the foam material and the open space created by the pore openings, also referred to as open-cells.

Thus, in an embodiment, a foam material has an open-cell surface. In another embodiment, a foam material has an open-cell surface having a surface porosity sufficient to allow tissue growth into the foam material.

In aspects of this embodiment, a foam material having an open-cell surface has a surface porosity of about 40% of the total surface area of the foam material, about 50% of the total surface area of, e.g., the foam material, about 60% of the total surface area of the foam material, about 70% of the total surface area of the foam material, about 80% of the total surface area of the foam material, about 90% of the total surface area of the foam material, about 95% of the total surface area of the foam material, or about 97% of the total surface area of the foam material. In other aspects of this embodiment, a foam material having an open-cell surface has a surface porosity of, e.g., at least 40% of the total surface area of the foam material, at least 50% of the total surface area of the foam material, at least 60% of the total surface area of the foam material, at least 70% of the total surface area of the foam material, at least 80% of the total surface area of the foam material, at least 90% of the total surface area of the foam material, at least 95% of the total surface area of the foam material, or at least 97% of the total surface area of the foam material. In yet other aspects of this embodiment, a foam material having an open-cell surface has a surface porosity of, e.g., at most 40% of the total surface area of the foam material, at most 50% of the total surface area of the foam material, at most 60% of the total surface area of the foam material, at most 70% of the total surface area of the foam material, at most 80% of the total surface area of the foam material, at most 90% of the total surface area of the foam material, at most 95% of the total surface area of the foam material, or at most 97% of the total surface area of the foam material. In still other aspects of this embodiment, a foam material having an open-cell surface has a surface porosity of, e.g., about 40% to about 90% of the total surface area of the foam material, about 50% to about 90% of the total surface area of the foam material, about 60% to about 90% of the total surface area of the foam material, about 70% to about 90% of the total surface area of the foam material, about 30% to about 100% of the total surface area of the foam material, about 40% to about 100% of the total surface area of the foam material, about 50% to about 100% of the total surface area of the foam material, about 60% to about 100% of the total surface area of the foam material, about 70% to about 100% of the total surface area of the foam material, or about 80% to about 100% of the total surface area of the foam material.

In aspects of this embodiment, a foam material has an open-cell surface that comprises, e.g., about 10% of the total surface area of the foam material, about 20% of the total surface area of the foam material, about 30% of the total surface area of the foam material, about 40% of the total surface area of the foam material, about 50% of the total surface area of the foam material, about 60% of the total surface area of the foam material, about 70% of the total surface area of the foam material, about 80% of the total surface area of the foam material, about 90% of the total surface area of the foam material, or about 100% of the total surface area of the foam material. In other aspects of this embodiment, a foam material has an open-cell surface that comprises, e.g., at least 10% of the total surface area of the foam material, at least 20% of the total surface area of the foam material, at least 30% of the total surface area of the foam material, at least 40% of the total surface area of the foam material, at least 50% of the total surface area of the foam material, at least 60% of the total surface area of the foam material, at least 70% of the total surface area of the foam material, at least 80% of the total surface area of the foam material, at least 90% of the total surface area of the foam material, or at least 95% of the total surface area of the foam material. In yet other aspects of this embodiment, a foam material has an open-cell surface that comprises, e.g., at most 10% of the total surface area of the foam material, at most 20% of the total surface area of the foam material, at most 30% of the total surface area of the foam material, at most 40% of the total surface area of the foam material, at most 50% of the total surface area of the foam material, at most 60% of the total surface area of the foam material, at most 70% of the total surface area of the foam material, at most 80% of the total surface area of the foam material, at most 90% of the total surface area of the foam material, or at most 95% of the total surface area of the foam material. In still other aspects of this embodiment, a foam material has an open-cell surface that comprises, e.g., about 30% to about 90% of the total surface area of the foam material, about 40% to about 90% of the total surface area of the foam material, about 50% to about 90% of the total surface area of the foam material, about 60% to about 90% of the total surface area of the foam material, about 70% to about 90% of the total surface area of the foam material, about 30% to about 100% of the total surface area of the foam material, about 40% to about 100% of the total surface area of the foam material, about 50% to about 100% of the total surface area of the foam material, about 60% to about 100% of the total surface area of the foam material, about 70% to about 100% of the total surface area of the foam material, or about 80% to about 100% of the total surface area of the foam material.

The present specification discloses, in part, an open-cell surface foam material comprising a substantially non-degradable, biocompatible, elastomer matrix. As used herein, the term “non-degradable” refers to a foam material that is not prone to degrading, decomposing, or breaking down to any substantial or significant degree while implanted in the host. Non-limiting examples of substantial non-degradation include less than 10% degradation of the foam material over the time period measured, less than 5% degradation of the foam material over the time period measured, less than 3% degradation of the foam material over the time period measured, less than 1% degradation of the foam material over the time period measured. As used herein, the term “biocompatible” refers to the ability of the foam material to perform its intended function, with the desired degree of incorporation in the host, without eliciting any undesirable local or systemic effects in that host.

In an embodiment, an open-cell surface foam material comprising an elastomer matrix defining an array of interconnected pores is substantially non-degradable. In aspects of this embodiment, an open-cell surface foam material comprising an elastomer matrix defining an array of interconnected pores is substantially non-degradable for, e.g., at least five years, at least ten years, at least 15 years, at least 20 years, at least 25 years, at least 30 years, at least 35 years, at least 40 years, at least 45 years, or at least 50 years. In yet other aspects of this embodiment, an open-cell surface foam material comprising an elastomer matrix defining an array of interconnected pores exhibits less than 5% degradation, less than 3% degradation, or less than 1% degradation over for, e.g., about five years, about ten years, about 15 years, about 20 years, about 25 years, about 30 years, about 35 years, about 40 years, about 45 years, or about 50 years. In still other aspects of this embodiment, an open-cell surface foam material comprising an elastomer matrix defining an array of interconnected pores exhibits less than 5% degradation, less than 3% degradation, or less than 1% degradation over for, e.g., at least five years, at least ten years, at least 15 years, at least 20 years, at least 25 years, at least 30 years, at least 35 years, at least 40 years, at least 45 years, or at least 50 years.

In another embodiment, an open-cell surface foam material comprising an elastomer matrix defining an array of interconnected pores is substantially biocompatible. In aspects of this embodiment, an open-cell surface foam material comprising an elastomer matrix defining an array of interconnected pores is substantially biocompatible for, e.g., at least five years, at least ten years, at least 15 years, at least 20 years, at least 25 years, at least 30 years, at least 35 years, at least 40 years, at least 45 years, or at least 50 years.

As used herein, the term “elastomer” or “elastic polymer” refers to an amorphous polymer capable of forming a foam that exists above its glass transition temperature (T_{g}) at ambient temperatures, thereby conferring the property of viscoelasticity so that considerable segmental motion is possible, and includes, without limitation, carbon-based elastomers, silicon-based elastomers, thermoset elastomers, and thermoplastic elastomers. As used herein, the term “ambient temperature” refers to a temperature of about 18° C. to about 22° C. Elastomers, either naturally-occurring or synthetically-made, comprise monomers usually made of carbon, hydrogen, oxygen, and/or silicon which are linked together to form long polymer chains. Elastomers are typically covalently cross-linked to one another, although non-covalently cross-linked elastomers are known. Elastomers may be homopolymers or copolymers, degradable, substantially non-degradable, or non-degradable. Copolymers may be random copolymers, blocked copolymers, graft copolymers, and/or mixtures thereof. Unlike other polymers classes, elastomers can be stretched many times its original length without breaking by reconfiguring themselves to distribute an applied stress, and the cross-linkages ensure that the elastomers will return to their original configuration when the stress is removed. Elastomers can be a non-medical grade elastomer or a medical grade elastomer. Medical grade elastomers are typically divided into three categories: non implantable, short term implantable and long-term implantable. Exemplary substantially non-degradable and/or non-degradable, biocompatible, elastomers include, without limitation, bromo isobutylene isoprene (BIIR), polybutadiene (BR), chloro isobutylene isoprene (CIIR), polychloroprene (CR), chlorosulphonated polyethylene (CSM), ethylene propylene (EP), ethylene propylene diene monomer (EPDM), fluoronated hydrocarbon (FKM), fluoro silicone (FVQM), hydrogenated nitrile butadiene (HNBR), polyisoprene (IR), isobutylene isoprene butyl (IIR), methyl vinyl silicone (MVQ), acrylonitrile butadiene (NBR), polyurethane (PU), styrene butadiene (SBR), styrene ethylene/butylene styrene (SEBS), polydimethylsiloxane (PDMS), polysiloxane (SI), and acrylonitrile butadiene carboxy monomer (XNBR).

The present specification discloses, in part, an elastomer that is a silicon-based elastomer. As used herein, the term “silicon-based elastomer” refers to any silicon containing elastomer capable of forming a foam, such as, e.g., methyl vinyl silicone, polydimethylsiloxane, or polysiloxane. A silicone-based elastomer can be a high temperature vulcanization (HTV) silicone or a room temperature vulcanization (RTV). A silicon-based elastomer can be a non-medical grade silicon-based elastomer or a medical grade silicon-based elastomer. As used herein, the term “medical grade silicon-based elastomer” refers to a silicon-based elastomer approved by the U.S. Pharmacopedia (USP) as at least Class V. Medical grade silicon-based elastomers are typically divided into three categories: non implantable, short term implantable and long-term implantable.

Thus, in an embodiment, an open-cell surface foam material comprises an elastomer matrix comprising a medical grade elastomer. In aspects of this embodiment, an open-cell surface foam material comprises an elastomer matrix that is, e.g., a medical grade carbon-based elastomer, a medical grade silicon-based elastomer, a medical grade thermoset elastomer, or a medical grade thermoplastic elastomer. In other aspects of this embodiment, an open-cell surface foam material comprises an elastomer matrix that is, e.g., a medical grade, long-term implantable, carbon-based elastomer, a medical grade, long-term implantable, silicon-based elastomer, a medical grade, long-term implantable, thermoset elastomer, or a medical grade, long-term implantable, thermoplastic elastomer. In still other aspects, an open-cell surface foam material comprising an elastomer matrix that is, e.g., a medical grade bromo isobutylene isoprene, a medical grade polybutadiene, a medical grade chloro isobutylene isoprene, a medical grade polychloroprene, a medical grade chlorosulphonated polyethylene, a medical grade ethylene propylene, a medical grade ethylene propylene diene monomer, a medical grade fluoronated hydrocarbon, a medical grade fluoro silicone, a medical grade hydrogenated nitrile butadiene, a medical grade polyisoprene, a medical grade isobutylene isoprene butyl, a medical grade methyl vinyl silicone, a medical grade acrylonitrile butadiene, a medical grade polyurethane, a medical grade styrene butadiene, a medical grade styrene ethylene/butylene styrene, a medical grade polydimethylsiloxane, a medical grade polysiloxane, or a medical grade acrylonitrile butadiene carboxy monomer.

In another embodiment, an open-cell surface foam material comprising an elastomer matrix that is a silicon-based elastomer. In an aspect of this embodiment, an open-cell surface foam material comprising an elastomer matrix that is a medical grade silicon-based elastomer. In aspects of this embodiment, an open-cell surface foam material comprising an elastomer matrix that is, e.g., at least a USP Class V silicon-based elastomer, at least a USP Class VI silicon-based elastomer, or USP Class VII silicon-based elastomer. In yet other aspects, an open-cell surface foam material comprising an elastomer matrix that is a long-term implantable silicon-based elastomer. In yet other aspects, an open-cell surface foam material comprising an elastomer matrix that is, e.g., a medical grade, long-term implantable, methyl vinyl silicone, a medical grade, long-term implantable, polydimethylsiloxane, or a medical grade, long-term implantable, polysiloxane.

Elastomers have the property of viscoelasticity. Viscoelasticity is the property of materials that exhibit both viscous and elastic characteristics when undergoing deformation. Viscous materials resist shear flow and strain linearly with time when a stress is applied. Elastic materials strain instantaneously when stretched and just as quickly return to their original state once the stress is removed. Viscoelastic materials have elements of both of these properties and, as such, exhibit time dependent strain. A viscoelastic material has the following properties: 1) hysteresis, or memory, is seen in the stress-strain curve; 2) stress relaxation occurs: step constant strain causes decreasing stress; and 3) creep occurs: step constant stress causes increasing strain. The viscoelasticity of elastomers confer a unique set of properties involving elongation, tensile strength, shear strength compressive modulus, and hardness that distinguish elastomers form other classes of polymers.

The present specification discloses, in part, an open-cell surface foam material comprising an elastomer matrix defining an array of interconnected pores. As used herein, the term “matrix” or “elastomer matrix” is synonymous with “cured elastomer” and refers to a three-dimensional structural framework composed of a substantially non-degradable, biocompatible elastomer in its cured state. As used herein, the term “silicon-based elastomer matrix” is synonymous with “cured silicon-based elastomer” and refers to a three-dimensional structural framework composed of a substantially non-degradable, biocompatible silicon-based elastomer in its cured state.

An open-cell surface foam material comprising an elastomer matrix defining an array of interconnected pores exhibits high resistance to deformation. Resistance to deformation is the ability of a foam material to maintain its original form after being exposed to stress, and can be calculated as the original form of a foam material (L₀), divided by the form of a foam material after it is released from a stress (L_{R}), and then multiplied by 100.

In an embodiment, an open-cell surface foam material comprising an elastomer matrix defining an array of interconnected pores exhibits high resistance to deformation. In aspects of this embodiment, an open-cell surface foam material comprising an elastomer matrix defining an array of interconnected pores exhibits resistance to deformation of, e.g., about 100%, about 99%, about 98%, about 97%, about 96%, about 95%, about 94%, about 93%, about 92%, about 91%, about 90%, about 89%, about 88%, about 87%, about 86%, or about 85%. In other aspects of this embodiment, an open-cell surface foam material comprising an elastomer matrix defining an array of interconnected pores exhibits resistance to deformation of, e.g., at least 99%, at least 98%, at least 97%, at least 96%, at least 95%, at least 94%, at least 93%, at least 92%, at least 91%, at least 90%, at least 89%, at least 88%, at least 87%, at least 86%, or at least 85%. In yet other aspects of this embodiment, an open-cell surface foam material comprising an elastomer matrix defining an array of interconnected pores exhibits resistance to deformation of, e.g., at most 99%, at most 98%, at most 97%, at most 96%, at most 95%, at most 94%, at most 93%, at most 92%, at most 91%, at most 90%, at most 89%, at most 88%, at most 87%, at most 86%, or at most 85%. In still aspects of this embodiment, an open-cell surface foam material comprising an elastomer matrix defining an array of interconnected pores exhibits resistance to deformation of, e.g., about 85% to about 100%, about 87% to about 100%, about 90% to about 100%, about 93% to about 100%, about 95% to about 100%, or about 97% to about 100%.

An open-cell surface foam material comprising an elastomer matrix elastomer matrix defining an array of interconnected pores exhibits high elastic elongation. Elongation is a type of deformation caused when an elastomer stretches under a tensile stress. Deformation is simply a change in shape that anything undergoes under stress. The elongation property of a foam material can be expressed as percent elongation, which is calculated as the length of a foam material after it is stretched (L), divided by the original length of the foam material (L₀), and then multiplied by 100. In addition, this elastic elongation may be reversible. Reversible elongation is the ability of a foam material to return to its original length after being release for a tensile stress, and can be calculated as the original length of a foam material (L₀), divided by the length of a foam material after it is released from a tensile stress (L_{R}), and then multiplied by 100.

In an embodiment, an open-cell surface foam material comprising an elastomer matrix defining an array of interconnected pores exhibits high elastic elongation. In aspects of this embodiment, an open-cell surface foam material comprising an elastomer matrix defining an array of interconnected pores exhibits an elastic elongation of, e.g., about 50%, about 100%, about 200%, about 300%, about 400%, about 500%, about 600%, about 700%, about 800%, about 900%, about 1000%, about 1100%, about 1200%, about 1300%, about 1400%, about 1500%, about 1600%, about 1700%, about 1800%, about 1900%, or about 2000%. In other aspects of this embodiment, an open-cell surface foam material comprising an elastomer matrix defining an array of interconnected pores exhibits an elastic elongation of, e.g., at least 50%, at least 100%, at least 200%, at least 300%, at least 400%, at least 500%, at least 600%, at least 700%, at least 800%, at least 900%, at least 1000%, at least 1100%, at least 1200%, at least 1300%, at least 1400%, at least 1500%, at least 1600%, at least 1700%, at least 1800%, at least 1900%, or at least 2000%. In yet other aspects of this embodiment, an open-cell surface foam material comprising an elastomer matrix defining an array of interconnected pores exhibits an elastic elongation of, e.g., at most 50%, at most 100%, at most 200%, at most 300%, at most 400%, at most 500%, at most 600%, at most 700%, at most 800%, at most 900%, at most 1000%, at most 1100%, at most 1200%, at most 1300%, at most 1400%, at most 1500%, at most 1600%, at most 1700%, at most 1800%, at most 1900%, or at most 2000%. In still aspects of this embodiment, an open-cell surface foam material comprising an elastomer matrix defining an array of interconnected pores exhibits an elastic elongation of, e.g., about 50% to about 600%, about 50% to about 700%, about 50% to about 800%, about 50% to about 900%, about 50% to about 1000%, about 100% to about 600%, about 100% to about 700%, about 100% to about 800%, about 100% to about 900%, about 100% to about 1000%, about 200% to about 600%, about 200% to about 700%, about 200% to about 800%, about 200% to about 900%, or about 200% to about 1000%.

In another embodiment, an open-cell surface foam material comprising an elastomer matrix defining an array of interconnected pores exhibits reversible elongation. In aspects of this embodiment, an open-cell surface foam material comprising an elastomer matrix defining an array of interconnected pores exhibits a reversible elastic elongation of, e.g., about 100%, about 99%, about 98%, about 97%, about 96%, about 95%, about 94%, about 93%, about 92%, about 91%, about 90%, about 89%, about 88%, about 87%, about 86%, or about 85%. In other aspects of this embodiment, an open-cell surface foam material comprising an elastomer matrix defining an array of interconnected pores exhibits a reversible elastic elongation of, e.g., at least 99%, at least 98%, at least 97%, at least 96%, at least 95%, at least 94%, at least 93%, at least 92%, at least 91%, at least 90%, at least 89%, at least 88%, at least 87%, at least 86%, or at least 85%. In yet other aspects of this embodiment, an open-cell surface foam material comprising an elastomer matrix defining an array of interconnected pores exhibits a reversible elastic elongation of, e.g., at most 99%, at most 98%, at most 97%, at most 96%, at most 95%, at most 94%, at most 93%, at most 92%, at most 91%, at most 90%, at most 89%, at most 88%, at most 87%, at most 86%, or at most 85%. In still aspects of this embodiment, an open-cell surface foam material comprising an elastomer matrix defining an array of interconnected pores exhibits a reversible elastic elongation of, e.g., about 85% to about 100%, about 87% to about 100%, about 90% to about 100%, about 93% to about 100%, about 95% to about 100%, or about 97% to about 100%.

An open-cell surface foam material comprising an elastomer matrix defining an array of interconnected pores exhibits low elastic modulus. Elastic modulus, or modulus of elasticity, refers to the ability of a foam material to resists deformation, or, conversely, an object's tendency to be non-permanently deformed when a force is applied to it. The elastic modulus of an object is defined as the slope of its stress-strain curve in the elastic deformation region: λ=stress/strain, where λ is the elastic modulus in Pascal's; stress is the force causing the deformation divided by the area to which the force is applied; and strain is the ratio of the change caused by the stress to the original state of the object. Specifying how stresses are to be measured, including directions, allows for many types of elastic moduli to be defined. The three primary elastic moduli are tensile modulus, shear modulus, and bulk modulus.

Tensile modulus (E) or Young's modulus is an objects response to linear strain, or the tendency of an object to deform along an axis when opposing forces are applied along that axis. It is defined as the ratio of tensile stress to tensile strain. It is often referred to simply as the elastic modulus. The shear modulus or modulus of rigidity refers to an object's tendency to shear (the deformation of shape at constant volume) when acted upon by opposing forces. It is defined as shear stress over shear strain. The shear modulus is part of the derivation of viscosity. The shear modulus is concerned with the deformation of a solid when it experiences a force parallel to one of its surfaces while its opposite face experiences an opposing force (such as friction). The bulk modulus (K) describes volumetric elasticity or an object's resistance to uniform compression, and is the tendency of an object to deform in all directions when uniformly loaded in all directions. It is defined as volumetric stress over volumetric strain, and is the inverse of compressibility. The bulk modulus is an extension of Young's modulus to three dimensions.

In another embodiment, an open-cell surface foam material comprising an elastomer matrix defining an array of interconnected pores exhibits low tensile modulus. In aspects of this embodiment, an open-cell surface foam material comprising an elastomer matrix defining an array of interconnected pores exhibits a tensile modulus of, e.g., about 0.01 MPa, about 0.02 MPa, about 0.03 MPa, about 0.04 MPa, about 0.05 MPa, about 0.06 MPa, about 0.07 MPa, about 0.08 MPa, about 0.09 MPa, about 0.1 MPa, about 0.15 MPa, about 0.2 MPa, about 0.25 MPa, about 0.3 MPa, about 0.35 MPa, about 0.4 MPa, about 0.45 MPa, about 0.5 MPa, about 0.55 MPa, about 0.6 MPa, about 0.65 MPa, or about 0.7 MPa. In other aspects of this embodiment, an open-cell surface foam material comprising an elastomer matrix defining an array of interconnected pores exhibits a tensile modulus of, e.g., at most 0.01 MPa, at most 0.02 MPa, at most 0.03 MPa, at most 0.04 MPa, at most 0.05 MPa, at most 0.06 MPa, at most 0.07 MPa, at most 0.08 MPa, at most 0.09 MPa, at most 0.1 MPa, at most 0.15 MPa, at most 0.2 MPa, at most 0.25 MPa, at most 0.3 MPa, at most 0.35 MPa, at most 0.4 MPa, at most 0.45 MPa, at most 0.5 MPa, at most 0.55 MPa, at most 0.6 MPa, at most 0.65 MPa, or at most 0.7 MPa. In yet other aspects of this embodiment, an open-cell surface foam material comprising an elastomer matrix defining an array of interconnected pores exhibits a tensile modulus of, e.g., about 0.01 MPa to about 0.1 MPa, about 0.01 MPa to about 0.2 MPa, about 0.01 MPa to about 0.3 MPa, about 0.01 MPa to about 0.4 MPa, about 0.01 MPa to about 0.5 MPa, about 0.01 MPa to about 0.6 MPa, or about 0.01 MPa to about 0.7 MPa.

In another embodiment, an open-cell surface foam material comprising an elastomer matrix defining an array of interconnected pores exhibits low shear modulus. In aspects of this embodiment, an open-cell surface foam material comprising an elastomer matrix defining an array of interconnected pores exhibits a shear modulus of, e.g., about 0.1 MPa, about 0.2 MPa, about 0.3 MPa, about 0.4 MPa, about 0.5 MPa, about 0.6 MPa, about 0.7 MPa, about 0.8 MPa, about 0.9 MPa, about 1 MPa, about 1.5 MPa, about 2 MPa, about 2.5 MPa, or about 3 MPa. In other aspects of this embodiment, an open-cell surface foam material comprising an elastomer matrix defining an array of interconnected pores exhibits a shear modulus of, e.g., at most 0.1 MPa, at most 0.2 MPa, at most 0.3 MPa, at most 0.4 MPa, at most 0.5 MPa, at most 0.6 MPa, at most 0.7 MPa, at most 0.8 MPa, at most 0.9 MPa, at most 1 MPa, at most 1.5 MPa, at most 2 MPa, at most 2.5 MPa, or at most 3 MPa. In yet other aspects of this embodiment, an open-cell surface foam material comprising an elastomer matrix defining an array of interconnected pores exhibits a shear modulus of, e.g., about 0.1 MPa to about 1 MPa, about 0.1 MPa to about 1.5 MPa, about 0.1 MPa to about 2 MPa, about 0.1 MPa to about 2.5 MPa, or about 0.1 MPa to about 3 MPa.

In another embodiment, an open-cell surface foam material comprising an elastomer matrix defining an array of interconnected pores exhibits low bulk modulus. In aspects of this embodiment, an open-cell surface foam material comprising an elastomer matrix defining an array of interconnected pores exhibits a bulk modulus of, e.g., about 0.5 GPa, about 0.6 GPa, about 0.7 GPa, about 0.8 GPa, about 0.9 GPa, about 1 GPa, about 1.5 GPa, about 2 GPa, about 2.5 GPa, about 3 GPa, about 3.5 GPa, about 4 GPa, about 4.5 GPa, or about 5 GPa. In other aspects of this embodiment, an open-cell surface foam material comprising an elastomer matrix defining an array of interconnected pores exhibits a bulk modulus of, e.g., at most 0.5 GPa, at most 0.6 GPa, at most 0.7 GPa, at most 0.8 GPa, at most 0.9 GPa, at most 1 GPa, at most 1.5 GPa, at most 2 GPa, at most 2.5 GPa, at most 3 GPa, at most 3.5 GPa, at most 4 GPa, at most 4.5 GPa, or at most 5 GPa. In yet other aspects of this embodiment, an open-cell surface foam material comprising an elastomer matrix defining an array of interconnected pores exhibits a bulk modulus of, e.g., about 0.5 GPa to about 5 GPa, about 0.5 GPa to about 1 GPa, or about 1 GPa to about 5 GPa.

An open-cell surface foam material comprising an elastomer matrix defining an array of interconnected pores exhibits high tensile strength relative to other polymer classes. Other polymer classes include any other polymer not classified as an elastomer. Tensile strength has three different definitional points of stress maxima. Yield strength refers to the stress at which material strain changes from elastic deformation to plastic deformation, causing it to deform permanently. Ultimate strength refers to the maximum stress a material can withstand when subjected to tension, compression or shearing. It is the maximum stress on the stress-strain curve. Breaking strength refers to the stress coordinate on the stress-strain curve at the point of rupture, or when the material pulls apart.

In another embodiment, an open-cell surface foam material comprising an elastomer matrix defining an array of interconnected pores exhibits high yield strength relative to other polymer classes. In aspects of this embodiment, an open-cell surface foam material comprising an elastomer matrix defining an array of interconnected pores exhibits a yield strength of, e.g., about 1 MPa, about 5 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa, about 600 MPa, about 700 MPa, about 800 MPa, about 900 MPa, about 1000 MPa, about 1500 MPa, or about 2000 MPa. In other aspects of this embodiment, an open-cell surface foam material comprising an elastomer matrix defining an array of interconnected pores exhibits a yield strength of, e.g., at least 1 MPa, at least 5 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa, at least 600 MPa, at least 700 MPa, at least 800 MPa, at least 900 MPa, at least 1000 MPa, at least 1500 MPa, or at least 2000 MPa. In yet other aspects of this embodiment, an open-cell surface foam material comprising an elastomer matrix defining an array of interconnected pores exhibits a yield strength of, e.g., at most 1 MPa, at most 5 MPa, at most 10 MPa, at most 20 MPa, at most 30 MPa, at most 40 MPa, at most 50 MPa, at most 60 MPa, at most 70 MPa, at most 80 MPa, at most 90 MPa, at most 100 MPa, at most 200 MPa, at most 300 MPa, at most 400 MPa, at most 500 MPa, at most 600 MPa, at most 700 MPa, at most 800 MPa, at most 900 MPa, at most 1000 MPa, at most 1500 MPa, or at most 2000 MPa. In still other aspects of this embodiment, an open-cell surface foam material comprising an elastomer matrix defining an array of interconnected pores exhibits a yield strength of, e.g., about 1 MPa to about 50 MPa, about 1 MPa to about 60 MPa, about 1 MPa to about 70 MPa, about 1 MPa to about 80 MPa, about 1 MPa to about 90 MPa, about 1 MPa to about 100 MPa, about 10 MPa to about 50 MPa, about 10 MPa to about 60 MPa, about 10 MPa to about 70 MPa, about 10 MPa to about 80 MPa, about 10 MPa to about 90 MPa, about 10 MPa to about 100 MPa, about 100 MPa to about 500 MPA, about 300 MPa to about 500 MPA, about 300 MPa to about 1000 MPa, about 500 MPa to about 1000 MPa, about 700 MPa to about 1000 MPa, about 700 MPa to about 1500 MPa, about 1000 MPa to about 1500 MPa, or about 1200 MPa to about 1500 MPa.

In another embodiment, an open-cell surface foam material comprising an elastomer matrix defining an array of interconnected pores exhibits high ultimate strength relative to other polymer classes. In aspects of this embodiment, an open-cell surface foam material comprising an elastomer matrix defining an array of interconnected pores exhibits an ultimate strength of, e.g., about 1 MPa, about 5 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa, about 600 MPa, about 700 MPa, about 800 MPa, about 900 MPa, about 1000 MPa, about 1500 MPa, or about 2000 MPa. In other aspects of this embodiment, an open-cell surface foam material comprising an elastomer matrix defining an array of interconnected pores exhibits an ultimate strength of, e.g., at least 1 MPa, at least 5 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa, at least 600 MPa, at least 700 MPa, at least 800 MPa, at least 900 MPa, at least 1000 MPa, at least 1500 MPa, or at least 2000 MPa. In yet other aspects of this embodiment, an open-cell surface foam material comprising an elastomer matrix defining an array of interconnected pores exhibits an ultimate strength of, e.g., at most 1 MPa, at most 5 MPa, at most 10 MPa, at most 20 MPa, at most 30 MPa, at most 40 MPa, at most 50 MPa, at most 60 MPa, at most 70 MPa, at most 80 MPa, at most 90 MPa, at most 100 MPa, at most 200 MPa, at most 300 MPa, at most 400 MPa, at most 500 MPa, at most 600 MPa, at most 700 MPa, at most 800 MPa, at most 900 MPa, at most 1000 MPa, at most 1500 MPa, or at most 2000 MPa. In still other aspects of this embodiment, an open-cell surface foam material comprising an elastomer matrix defining an array of interconnected pores exhibits an ultimate strength of, e.g., about 1 MPa to about 50 MPa, about 1 MPa to about 60 MPa, about 1 MPa to about 70 MPa, about 1 MPa to about 80 MPa, about 1 MPa to about 90 MPa, about 1 MPa to about 100 MPa, about 10 MPa to about 50 MPa, about 10 MPa to about 60 MPa, about 10 MPa to about 70 MPa, about 10 MPa to about 80 MPa, about 10 MPa to about 90 MPa, about 10 MPa to about 100 MPa, about 100 MPa to about 500 MPA, about 300 MPa to about 500 MPA, about 300 MPa to about 1000 MPa, about 500 MPa to about 1000 MPa, about 700 MPa to about 1000 MPa, about 700 MPa to about 1500 MPa, about 1000 MPa to about 1500 MPa, or about 1200 MPa to about 1500 MPa.

In another embodiment, an open-cell surface foam material comprising an elastomer matrix defining an array of interconnected pores exhibits high breaking strength relative to other polymer classes. In aspects of this embodiment, an open-cell surface foam material comprising an elastomer matrix defining an array of interconnected pores exhibits a breaking strength of, e.g., about 1 MPa, about 5 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa, about 600 MPa, about 700 MPa, about 800 MPa, about 900 MPa, about 1000 MPa, about 1500 MPa, or about 2000 MPa. In other aspects of this embodiment, an open-cell surface foam material comprising an elastomer matrix defining an array of interconnected pores exhibits a breaking strength of, e.g., at least 1 MPa, at least 5 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa, at least 600 MPa, at least 700 MPa, at least 800 MPa, at least 900 MPa, at least 1000 MPa, at least 1500 MPa, or at least 2000 MPa. In yet other aspects of this embodiment, an open-cell surface foam material comprising an elastomer matrix defining an array of interconnected pores exhibits a breaking strength of, e.g., at most 1 MPa, at most 5 MPa, at most 10 MPa, at most 20 MPa, at most 30 MPa, at most 40 MPa, at most 50 MPa, at most 60 MPa, at most 70 MPa, at most 80 MPa, at most 90 MPa, at most 100 MPa, at most 200 MPa, at most 300 MPa, at most 400 MPa, at most 500 MPa, at most 600 MPa, at most 700 MPa, at most 800 MPa, at most 900 MPa, at most 1000 MPa, at most 1500 MPa, or at most 2000 MPa. In still other aspects of this embodiment, an open-cell surface foam material comprising an elastomer matrix defining an array of interconnected pores exhibits a breaking strength of, e.g., about 1 MPa to about 50 MPa, about 1 MPa to about 60 MPa, about 1 MPa to about 70 MPa, about 1 MPa to about 80 MPa, about 1 MPa to about 90 MPa, about 1 MPa to about 100 MPa, about 10 MPa to about 50 MPa, about 10 MPa to about 60 MPa, about 10 MPa to about 70 MPa, about 10 MPa to about 80 MPa, about 10 MPa to about 90 MPa, about 10 MPa to about 100 MPa, about 100 MPa to about 500 MPA, about 300 MPa to about 500 MPA, about 300 MPa to about 1000 MPa, about 500 MPa to about 1000 MPa, about 700 MPa to about 1000 MPa, about 700 MPa to about 1500 MPa, about 1000 MPa to about 1500 MPa, or about 1200 MPa to about 1500 MPa.

An open-cell surface foam material comprising an elastomer matrix defining an array of interconnected pores exhibits low flexural strength relative to other polymer classes. Flexural strength, also known as bend strength or modulus of rupture, refers to an object's ability to resist deformation under load and represents the highest stress experienced within the object at its moment of rupture. It is measured in terms of stress.

In an embodiment, an open-cell surface foam material comprising an elastomer matrix defining an array of interconnected pores exhibits low flexural strength relative to other polymer classes. In aspects of this embodiment, an open-cell surface foam material comprising an elastomer matrix defining an array of interconnected pores exhibits a flexural strength of, e.g., about 1 MPa, about 5 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa, about 600 MPa, about 700 MPa, about 800 MPa, about 900 MPa, about 1000 MPa, about 1500 MPa, or about 2000 MPa. In other aspects of this embodiment, an open-cell surface foam material comprising an elastomer matrix defining an array of interconnected pores exhibits a flexural strength of, e.g., at least 1 MPa, at least 5 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa, at least 600 MPa, at least 700 MPa, at least 800 MPa, at least 900 MPa, at least 1000 MPa, at least 1500 MPa, or at least 2000 MPa. In yet other aspects of this embodiment, an open-cell surface foam material comprising an elastomer matrix defining an array of interconnected pores exhibits a flexural strength of, e.g., at most 1 MPa, at most 5 MPa, at most 10 MPa, at most 20 MPa, at most 30 MPa, at most 40 MPa, at most 50 MPa, at most 60 MPa, at most 70 MPa, at most 80 MPa, at most 90 MPa, at most 100 MPa, at most 200 MPa, at most 300 MPa, at most 400 MPa, at most 500 MPa, at most 600 MPa, at most 700 MPa, at most 800 MPa, at most 900 MPa, at most 1000 MPa, at most 1500 MPa, or at most 2000 MPa. In still other aspects of this embodiment, an open-cell surface foam material comprising an elastomer matrix defining an array of interconnected pores exhibits a flexural strength of, e.g., about 1 MPa to about 50 MPa, about 1 MPa to about 60 MPa, about 1 MPa to about 70 MPa, about 1 MPa to about 80 MPa, about 1 MPa to about 90 MPa, about 1 MPa to about 100 MPa, about 10 MPa to about 50 MPa, about 10 MPa to about 60 MPa, about 10 MPa to about 70 MPa, about 10 MPa to about 80 MPa, about 10 MPa to about 90 MPa, about 10 MPa to about 100 MPa, about 100 MPa to about 500 MPA, about 300 MPa to about 500 MPA, about 300 MPa to about 1000 MPa, about 500 MPa to about 1000 MPa, about 700 MPa to about 1000 MPa, about 700 MPa to about 1500 MPa, about 1000 MPa to about 1500 MPa, or about 1200 MPa to about 1500 MPa.

An open-cell surface foam material comprising an elastomer matrix defining an array of interconnected pores exhibits high compressibility. Compressibility refers to the relative volume change in response to a pressure (or mean stress) change, and is the reciprocal of the bulk modulus.

In an embodiment, an open-cell surface foam material comprising an elastomer matrix defining an array of interconnected pores exhibits high compressibility. In aspects of this embodiment, an open-cell surface foam material comprising an elastomer matrix defining an array of interconnected pores exhibits a compressibility of, e.g., about 0.1 kPa, about 0.5 kPa, about 1 kPa, about 5 kPa, about 10 kPa, about 15 kPa, about 20 kPa, about 30 kPa, about 40 kPa, about 50 kPa, about 60 kPa, about 70 kPa, about 80 kPa, about 90 kPa, or about 100 kPa. In other aspects of this embodiment, an open-cell surface foam material comprising an elastomer matrix defining an array of interconnected pores exhibits a compressibility of, e.g., at least 0.1 kPa, at least 0.5 kPa, at least 1 kPa, at least 5 kPa, at least 10 kPa, at least 15 kPa, at least 20 kPa, at least 30 kPa, at least 40 kPa, at least 50 kPa, at least 60 kPa, at least 70 kPa, at least 80 kPa, at least 90 kPa, or at least 100 kPa. In yet other aspects of this embodiment, an open-cell surface foam material comprising an elastomer matrix defining an array of interconnected pores exhibits a compressibility of, e.g., at most 0.1 kPa, at most 0.5 kPa, at most 1 kPa, at most 5 kPa, at most 10 kPa, at most 15 kPa, at most 20 kPa, at most 30 kPa, at most 40 kPa, at most 50 kPa, at most 60 kPa, at most 70 kPa, at most 80 kPa, at most 90 kPa, or at most 100 kPa. In still other aspects of this embodiment, an open-cell surface foam material comprising an elastomer matrix defining an array of interconnected pores exhibits a compressibility of, e.g., about 0.1 kPa to about 100 kPa, about 0.5 kPa to about 100 kPa, about 1 kPa to about 100 kPa, about 5 kPa to about 100 kPa, about 10 kPa to about 100 kPa, about 1 kPa to about 30 kPa, about 1 kPa to about 40 kPa, about 1 kPa to about 50 kPa, or about 1 kPa to about 60 kPa.

An open-cell surface foam material comprising an elastomer matrix defining an array of interconnected pores exhibits low hardness. Hardness refers to various properties of an object in the solid phase that gives it high resistance to various kinds of shape change when force is applied. Hardness is measured using a durometer and is a unitless value that ranges from zero to 100.

In an embodiment, an open-cell surface foam material comprising an elastomer matrix defining an array of interconnected pores exhibits low hardness. In aspects of this embodiment, an open-cell surface foam material comprising an elastomer matrix defining an array of interconnected pores exhibits a hardness of, e.g., about 5, about 10, about 15, about 20, about 25, about 30, about 35, about 40, about 45, about 50, about 55, or about 60. In other aspects of this embodiment, an open-cell surface foam material comprising an elastomer matrix defining an array of interconnected pores exhibits a hardness of, e.g., at least 5, at least 10, at least 15, at least 20, at least 25, at least 30, at least 35, at least 40, at least 45, at least 50, at least 55, or at least 60. In yet other aspects of this embodiment, an open-cell surface foam material comprising an elastomer matrix defining an array of interconnected pores exhibits a hardness of, e.g., at most 5, at most 10, at most 15, at most 20, at most 25, at most 30, at most 35, at most 40, at most 45, at most 50, at most 55, or at most 60. In still other aspects of this embodiment, an open-cell surface foam material comprising an elastomer matrix defining an array of interconnected pores exhibits a hardness of, e.g., about 5 to about 60, about 10 to about 50, about 15 to about 45, about 20 to about 40, or about 25 to about 35.

An open-cell surface foam material comprising an elastomer matrix includes pores having a shape sufficient to allow tissue growth into the array of interconnected pores. As such, the pore shape should support aspects of tissue growth such as, e.g., cell migration, cell proliferation, cell differentiation, nutrient exchange, and/or waste removal. Any pore shape is useful with the proviso that the pore shape is sufficient to allow tissue growth into the array of interconnected pores. Useful pore shapes include, without limited, roughly spherical, perfectly spherical, dodecahedrons (such as pentagonal dodecahedrons), and ellipsoids.

An open-cell surface foam material comprising an elastomer matrix includes pores having a roundness sufficient to allow tissue growth into the array of interconnected pores. As such, the pore roundness should support aspects of tissue growth such as, e.g., cell migration, cell proliferation, cell differentiation, nutrient exchange, and/or waste removal. As used herein, “roundness” is defined as (6×V)/(π×D³), where V is the volume and D is the diameter. Any pore roundness is useful with the proviso that the pore roundness is sufficient to allow tissue growth into the array of interconnected pores.

An open-cell surface foam material comprising an elastomer matrix is formed in such a manner that substantially all the pores in the elastomer matrix have a similar diameter. As used herein, the term “substantially”, when used to describe pores, refers to at least 90% of the pores comprising the elastomer matrix such as, e.g., at least 95% or at least 97% of the pores. As used herein, the term “similar diameter”, when used to describe pores, refers to a difference in the diameters of the two pores that is less than about 20% of the larger diameter. As used herein, the term “diameter”, when used to describe pores, refers to the longest line segment that can be drawn that connects two points within the pore, regardless of whether the line passes outside the boundary of the pore. Any pore diameter is useful with the proviso that the pore diameter is sufficient to allow tissue growth into the foam material. As such, the pore diameter size should support aspects of tissue growth such as, e.g., cell migration, cell proliferation, cell differentiation, nutrient exchange, and/or waste removal.

An open-cell surface foam material comprising an elastomer matrix is formed in such a manner that the diameter of the connections between pores is sufficient to allow tissue growth into the array of interconnected pores. As such, the diameter of the connections between pores should support aspects of tissue growth such as, e.g., cell migration, cell proliferation, cell differentiation, nutrient exchange, and/or waste removal. As used herein, the term “diameter”, when describing the connection between pores, refers to the diameter of the cross-section of the connection between two pores in the plane normal to the line connecting the centroids of the two pores, where the plane is chosen so that the area of the cross-section of the connection is at its minimum value. As used herein, the term “diameter of a cross-section of a connection” refers to the average length of a straight line segment that passes through the center, or centroid (in the case of a connection having a cross-section that lacks a center), of the cross-section of a connection and terminates at the periphery of the cross-section. As used herein, the term “substantially”, when used to describe the connections between pores refers to at least 90% of the connections made between each pore comprising the elastomer matrix, such as, e.g., at least 95% or at least 97% of the connections.

Thus, in an embodiment, an open-cell surface foam material comprising an elastomer matrix includes pores having a roundness sufficient to allow tissue growth into the array of interconnected pores. In aspects of this embodiment, an open-cell surface foam material comprising an elastomer matrix includes pores having a roundness of, e.g., about 0.1, about 0.2, about 0.3, about 0.4, about 0.5, about 0.6, about 0.7, about 0.8, about 0.9, or about 1.0. In other aspects of this embodiment, an open-cell surface foam material comprising an elastomer matrix includes pores having a roundness of, e.g., at least 0.1, at least 0.2, at least 0.3, at least 0.4, at least 0.5, at least 0.6, at least 0.7, at least 0.8, at least 0.9, or at least 1.0. In yet other aspects of this embodiment, an open-cell surface foam material comprising an elastomer matrix includes pores having a roundness of, e.g., at most 0.1, at most 0.2, at most 0.3, at most 0.4, at most 0.5, at most 0.6, at most 0.7, at most 0.8, at most 0.9, or at most 1.0. In still other aspects of this embodiment, an open-cell surface foam material comprising an elastomer matrix includes pores having a roundness of, e.g., about 0.1 to about 1.0, about 0.2 to about 1.0, about 0.3 to about 1.0, about 0.4 to about 1.0, about 0.5 to about 1.0, about 0.6 to about 1.0, about 0.7 to about 1.0, about 0.8 to about 1.0, about 0.9 to about 1.0, about 0.1 to about 0.9, about 0.2 to about 0.9, about 0.3 to about 0.9, about 0.4 to about 0.9, about 0.5 to about 0.9, about 0.6 to about 0.9, about 0.7 to about 0.9, about 0.8 to about 0.9, about 0.1 to about 0.8, about 0.2 to about 0.8, about 0.3 to about 0.8, about 0.4 to about 0.8, about 0.5 to about 0.8, about 0.6 to about 0.8, about 0.7 to about 0.8, about 0.1 to about 0.7, about 0.2 to about 0.7, about 0.3 to about 0.7, about 0.4 to about 0.7, about 0.5 to about 0.7, about 0.6 to about 0.7, about 0.1 to about 0.6, about 0.2 to about 0.6, about 0.3 to about 0.6, about 0.4 to about 0.6, about 0.5 to about 0.6, about 0.1 to about 0.5, about 0.2 to about 0.5, about 0.3 to about 0.5, or about 0.4 to about 0.5.

In another embodiment, substantially all pores within an open-cell surface foam material comprising an elastomer matrix have a similar diameter. In aspects of this embodiment, at least 90% of all pores within an open-cell surface foam material comprising an elastomer matrix have a similar diameter, at least 95% of all pores within an open-cell surface foam material comprising an elastomer matrix have a similar diameter, or at least 97% of all pores within an open-cell surface foam material comprising an elastomer matrix have a similar diameter. In another aspect of this embodiment, difference in the diameters of two pores is, e.g., less than about 20% of the larger diameter, less than about 15% of the larger diameter, less than about 10% of the larger diameter, or less than about 5% of the larger diameter.

In another embodiment, an open-cell surface foam material comprising an elastomer matrix has pores having a mean diameter sufficient to allow tissue growth into the array of interconnected pores. In aspects of this embodiment, an open-cell surface foam material comprising an elastomer matrix includes pores having mean pore diameter of, e.g., about 50 μm, about 75 μm, about 100 μm, about 150 μm, about 200 μm, about 250 μm, about 300 μm, about 350 μm, about 400 μm, about 450 μm, or about 500 μm. In other aspects, an open-cell surface foam material comprising an elastomer matrix includes pores having mean pore diameter of, e.g., about 500 μm, about 600 μm, about 700 μm, about 800 μm, about 900 μm, about 1000 μm, about 1500 μm, about 2000 μm, about 2500 μm, or about 3000 μm. In yet other aspects of this embodiment, an open-cell surface foam material comprising an elastomer matrix includes pores having mean pore diameter of, e.g., at least 50 μm, at least 75 μm, at least 100 μm, at least 150 μm, at least 200 μm, at least 250 μm, at least 300 μm, at least 350 μm, at least 400 μm, at least 450 μm, or at least 500 μm. In still other aspects, an open-cell surface foam material comprising an elastomer matrix includes pores having mean pore diameter of, e.g., at least 500 μm, at least 600 μm, at least 700 μm, at least 800 μm, at least 900 μm, at least 1000 μm, at least 1500 μm, at least 2000 μm, at least 2500 μm, or at least 3000 μm. In further aspects of this embodiment, an open-cell surface foam material comprising an elastomer matrix includes pores having mean pore diameter of, e.g., at most 50 μm, at most 75 μm, at most 100 μm, at most 150 μm, at most 200 μm, at most 250 μm, at most 300 μm, at most 350 μm, at most 400 μm, at most 450 μm, or at most 500 μm. In yet further aspects of this embodiment, an open-cell surface foam material comprising an elastomer matrix includes pores having mean pore diameter of, e.g., at most 500 μm, at most 600 μm, at most 700 μm, at most 800 μm, at most 900 μm, at most 1000 μm, at most 1500 μm, at most 2000 μm, at most 2500 μm, or at most 3000 μm. In still further aspects of this embodiment, an open-cell surface foam material comprising an elastomer matrix includes pores having mean pore diameter in a range from, e.g., about 300 μm to about 600 μm, about 200 μm to about 700 μm, about 100 μm to about 800 μm, about 500 μm to about 800 μm, about 50 μm to about 500 μm, about 75 μm to about 500 μm, about 100 μm to about 500 μm, about 200 μm to about 500 μm, about 300 μm to about 500 μm, about 50 μm to about 1000 μm, about 75 μm to about 1000 μm, about 100 μm to about 1000 μm, about 200 μm to about 1000 μm, about 300 μm to about 1000 μm, about 50 μm to about 1000 μm, about 75 μm to about 3000 μm, about 100 μm to about 3000 μm, about 200 μm to about 3000 μm, or about 300 μm to about 3000 μm.

In another embodiment, an open-cell surface foam material comprising an elastomer matrix includes pores having a mean elastomer strut thickness sufficient to allow tissue growth into the array of interconnected pores. In aspects of this embodiment, an open-cell surface foam material comprising an elastomer matrix includes pores having a mean elastomer strut thickness of, e.g., about 10 μm, about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, or about 200 μm. In other aspects of this embodiment, an open-cell surface foam material comprising an elastomer matrix includes pores having a mean elastomer strut thickness of, e.g., at least 10 μm, at least 20 μm, at least 30 μm, at least 40 μm, at least 50 μm, at least 60 μm, at least 70 μm, at least 80 μm, at least 90 μm, at least 100 μm, at least 110 μm, at least 120 μm, at least 130 μm, at least 140 μm, at least 150 μm, at least 160 μm, at least 170 μm, at least 180 μm, at least 190 μm, or at least 200 μm. In yet other aspects of this embodiment, an open-cell surface foam material comprising an elastomer matrix includes pores having a mean elastomer strut thickness of, e.g., at most 10 μm, at most 20 μm, at most 30 μm, at most 40 μm, at most 50 μm, at most 60 μm, at most 70 μm, at most 80 μm, at most 90 μm, at most 100 μm, at most 110 μm, at most 120 μm, at most 130 μm, at most 140 μm, at most 150 μm, at most 160 μm, at most 170 μm, at most 180 μm, at most 190 μm, or at most 200 μm. In still aspects of this embodiment, an open-cell surface foam material comprising an elastomer matrix includes pores having a mean elastomer strut thickness of, e.g., about 50 μm to about 110 μm, about 50 μm to about 120 μm, about 50 μm to about 130 μm, about 50 μm to about 140 μm, about 50 μm to about 150 μm, about 60 μm to about 110 μm, about 60 μm to about 120 μm, about 60 μm to about 130 μm, about 60 μm to about 140 μm, about 70 μm to about 110 μm, about 70 μm to about 120 μm, about 70 μm to about 130 μm, or about 70 μm to about 140 μm.

In another embodiment, an open-cell surface foam material comprising an elastomer matrix includes pores connected to a plurality of other pores. In aspects of this embodiment, an open-cell surface foam material comprising an elastomer matrix comprises a mean pore connectivity, e.g., about two other pores, about three other pores, about four other pores, about five other pores, about six other pores, about seven other pores, about eight other pores, about nine other pores, about ten other pores, about 11 other pores, or about 12 other pores. In other aspects of this embodiment, an open-cell surface foam material comprising an elastomer matrix comprises a mean pore connectivity, e.g., at least two other pores, at least three other pores, at least four other pores, at least five other pores, at least six other pores, at least seven other pores, at least eight other pores, at least nine other pores, at least ten other pores, at least 11 other pores, or at least 12 other pores. In yet other aspects of this embodiment, an open-cell surface foam material comprising an elastomer matrix comprises a mean pore connectivity, e.g., at most two other pores, at least most other pores, at least most other pores, at least most other pores, at most six other pores, at most seven other pores, at most eight other pores, at most nine other pores, at most ten other pores, at most 11 other pores, or at most 12 other pores.

In still other aspects of this embodiment, an open-cell surface foam material comprising an elastomer matrix includes pores connected to, e.g., about two other pores to about 12 other pores, about two other pores to about 11 other pores, about two other pores to about ten other pores, about two other pores to about nine other pores, about two other pores to about eight other pores, about two other pores to about seven other pores, about two other pores to about six other pores, about two other pores to about five other pores, about three other pores to about 12 other pores, about three other pores to about 11 other pores, about three other pores to about ten other pores, about three other pores to about nine other pores, about three other pores to about eight other pores, about three other pores to about seven other pores, about three other pores to about six other pores, about three other pores to about five other pores, about four other pores to about 12 other pores, about four other pores to about 11 other pores, about four other pores to about ten other pores, about four other pores to about nine other pores, about four other pores to about eight other pores, about four other pores to about seven other pores, about four other pores to about six other pores, about four other pores to about five other pores, about five other pores to about 12 other pores, about five other pores to about 11 other pores, about five other pores to about ten other pores, about five other pores to about nine other pores, about five other pores to about eight other pores, about five other pores to about seven other pores, or about five other pores to about six other pores.

In another embodiment, an open-cell surface foam material comprising an elastomer matrix includes pores where the diameter of the connections between pores is sufficient to allow tissue growth into the array of interconnected pores. In aspects of this embodiment, an open-cell surface foam material comprising an elastomer matrix includes pores where the diameter of the connections between pores is, e.g., about 10% the mean pore diameter, about 20% the mean pore diameter, about 30% the mean pore diameter, about 40% the mean pore diameter, about 50% the mean pore diameter, about 60% the mean pore diameter, about 70% the mean pore diameter, about 80% the mean pore diameter, or about 90% the mean pore diameter. In other aspects of this embodiment, an open-cell surface foam material comprising an elastomer matrix includes pores where the diameter of the connections between pores is, e.g., at least 10% the mean pore diameter, at least 20% the mean pore diameter, at least 30% the mean pore diameter, at least 40% the mean pore diameter, at least 50% the mean pore diameter, at least 60% the mean pore diameter, at least 70% the mean pore diameter, at least 80% the mean pore diameter, or at least 90% the mean pore diameter. In yet other aspects of this embodiment, an open-cell surface foam material comprising an elastomer matrix includes pores where the diameter of the connections between pores is, e.g., at most 10% the mean pore diameter, at most 20% the mean pore diameter, at most 30% the mean pore diameter, at most 40% the mean pore diameter, at most 50% the mean pore diameter, at most 60% the mean pore diameter, at most 70% the mean pore diameter, at most 80% the mean pore diameter, or at most 90% the mean pore diameter.

In still other aspects of this embodiment, an open-cell surface foam material comprising an elastomer matrix includes pores where the diameter of the connections between pores is, e.g., about 10% to about 90% the mean pore diameter, about 15% to about 90% the mean pore diameter, about 20% to about 90% the mean pore diameter, about 25% to about 90% the mean pore diameter, about 30% to about 90% the mean pore diameter, about 35% to about 90% the mean pore diameter, about 40% to about 90% the mean pore diameter, about 10% to about 80% the mean pore diameter, about 15% to about 80% the mean pore diameter, about 20% to about 80% the mean pore diameter, about 25% to about 80% the mean pore diameter, about 30% to about 80% the mean pore diameter, about 35% to about 80% the mean pore diameter, about 40% to about 80% the mean pore diameter, about 10% to about 70% the mean pore diameter, about 15% to about 70% the mean pore diameter, about 20% to about 70% the mean pore diameter, about 25% to about 70% the mean pore diameter, about 30% to about 70% the mean pore diameter, about 35% to about 70% the mean pore diameter, about 40% to about 70% the mean pore diameter, about 10% to about 60% the mean pore diameter, about 15% to about 60% the mean pore diameter, about 20% to about 60% the mean pore diameter, about 25% to about 60% the mean pore diameter, about 30% to about 60% the mean pore diameter, about 35% to about 60% the mean pore diameter, about 40% to about 60% the mean pore diameter, about 10% to about 50% the mean pore diameter, about 15% to about 50% the mean pore diameter, about 20% to about 50% the mean pore diameter, about 25% to about 50% the mean pore diameter, about 30% to about 50% the mean pore diameter, about 10% to about 40% the mean pore diameter, about 15% to about 40% the mean pore diameter, about 20% to about 40% the mean pore diameter, about 25% to about 40% the mean pore diameter, or about 30% to about 40% the mean pore diameter.

The present specification discloses, in part, an open-cell surface foam material comprising an elastomer matrix defining an array of interconnected pores having a porosity that is sufficient to allow tissue growth into the array of interconnected pores as disclosed in the present specification. As such, the porosity should support aspects of tissue growth such as, e.g., cell migration, cell proliferation, cell differentiation, nutrient exchange, and/or waste removal. As used herein, the term “porosity” refers to the amount of void space in an open-cell surface foam material comprising an elastomer matrix. As such, the total volume of an open-cell surface foam material comprising an elastomer matrix disclosed in the present specification is based upon the elastomer space and the void space.

Thus, in an embodiment, an open-cell surface foam material comprising an elastomer matrix defining an array of interconnected pores has a porosity sufficient to allow tissue growth into the array of interconnected pores. In aspects of this embodiment, an open-cell surface foam material comprising an elastomer matrix comprises a porosity of, e.g., about 50% of the total volume of an elastomer matrix, about 60% of the total volume of an elastomer matrix, about 70% of the total volume of an elastomer matrix, about 80% of the total volume of an elastomer matrix, about 90% of the total volume of an elastomer matrix, about 95% of the total volume of an elastomer matrix, or about 97% of the total volume of an elastomer matrix. In other aspects of this embodiment, an open-cell surface foam material comprising an elastomer matrix comprises a porosity of, e.g., at least 50% of the total volume of an elastomer matrix, at least 60% of the total volume of an elastomer matrix, at least 70% of the total volume of an elastomer matrix, at least 80% of the total volume of an elastomer matrix, at least 90% of the total volume of an elastomer matrix, at least 95% of the total volume of an elastomer matrix, or at least 97% of the total volume of an elastomer matrix. In yet other aspects of this embodiment, an open-cell surface foam material comprising an elastomer matrix comprises a porosity of, e.g., at most 50% of the total volume of an elastomer matrix, at most 60% of the total volume of an elastomer matrix, at most 70% of the total volume of an elastomer matrix, at most 80% of the total volume of an elastomer matrix, at most 90% of the total volume of an elastomer matrix, at most 95% of the total volume of an elastomer matrix, or at most 97% of the total volume of an elastomer matrix. In yet other aspects of this embodiment, an open-cell surface foam material comprising an elastomer matrix comprises a porosity of, e.g., about 50% to about 97% of the total volume of an elastomer matrix, about 60% to about 97% of the total volume of an elastomer matrix, about 70% to about 97% of the total volume of an elastomer matrix, about 80% to about 97% of the total volume of an elastomer matrix, about 90% to about 97% of the total volume of an elastomer matrix, about 50% to about 95% of the total volume of an elastomer matrix, about 60% to about 95% of the total volume of an elastomer matrix, about 70% to about 95% of the total volume of an elastomer matrix, about 80% to about 95% of the total volume of an elastomer matrix, about 90% to about 95% of the total volume of an elastomer matrix, about 50% to about 90% of the total volume of an elastomer matrix, about 60% to about 90% of the total volume of an elastomer matrix, about 70% to about 90% of the total volume of an elastomer matrix, or about 80% to about 90% of the total volume of an elastomer matrix.

The present specification discloses, in part, an open-cell surface foam material comprising an elastomer matrix defining an array of interconnected pores having a mean open pore value and/or a mean closed pore value that is sufficient to allow tissue growth into the array of interconnected pores as disclosed in the present specification. As used herein, the term “mean open pore value” or “mean open pore” refers to the average number of pores that are connected to at least one other pore present in the elastomer matrix. As used herein, the term “mean closed pore value” or “mean closed pore” refers to the average number of pores that are not connected to any other pores present in the elastomer matrix.

Thus, in an embodiment, an open-cell surface foam material comprising an elastomer matrix defining an array of interconnected pores has a mean open pore value sufficient to allow tissue growth into the array of interconnected pores. In aspects of this embodiment, an open-cell surface foam material comprising an elastomer matrix has a mean open pore value of, e.g., about 70%, about 75%, about 80%, about 85%, about 90%, about 95%, or about 97%. In other aspects of this embodiment, an open-cell surface foam material comprising an elastomer matrix has a mean open pore value of, e.g., at least 70%, at least 75%, at least 80%, at least 85%, at least 90%, at least 95%, or at least 97%. In yet other aspects of this embodiment, an open-cell surface foam material comprising an elastomer matrix has a mean open pore value of, e.g., at most 70%, at most 75%, at most 80%, at most 85%, at most 90%, at most 95%, or at most 97%. In still aspects of this embodiment, an open-cell surface foam material comprising an elastomer matrix has a mean open pore value of, e.g., about 70% to about 90%, about 75% to about 90%, about 80% to about 90%, about 85% to about 90%, about 70% to about 95%, about 75% to about 95%, about 80% to about 95%, about 85% to about 95%, about 90% to about 95%, about 70% to about 97%, about 75% to about 97%, about 80% to about 97%, about 85% to about 97%, or about 90% to about 97%.

In another embodiment, an open-cell surface foam material comprising an elastomer matrix defining an array of interconnected pores has a mean closed pore value sufficient to allow tissue growth into the array of interconnected pores. In aspects of this embodiment, an open-cell surface foam material comprising an elastomer matrix has a mean closed pore value of, e.g., about 5%, about 10%, about 15%, or about 20%. In other aspects of this embodiment, an open-cell surface foam material comprising an elastomer matrix has a mean closed pore value of, e.g., about 5% or less, about 10% or less, about 15% or less, or about 20% or less. In yet other aspects of this embodiment, an open-cell surface foam material comprising an elastomer matrix has a mean closed pore value of, e.g., about 5% to about 10%, about 5% to about 15%, or about 5% to about 20%.

The present specification discloses, in part, an open-cell surface foam material comprising an elastomer matrix defining an array of interconnected pores having a void space that is sufficient to allow tissue growth into the array of interconnected pores. As such, the void space should support aspects of tissue growth such as, e.g., cell migration, cell proliferation, cell differentiation, nutrient exchange, and/or waste removal. The term “void space” when used in reference to a foam material refers to the actual or physical space in a foam material, i.e., the actual space not physically occupied by the material comprising the foam material. As such, the total volume of an open-cell surface foam material disclosed in the present specification is based upon the elastomer space and the void space.

Thus, in an embodiment, an open-cell surface foam material comprising an elastomer matrix defining an array of interconnected pores has a void space sufficient to allow tissue growth into the array of interconnected pores. In aspects of this embodiment, an open-cell surface foam material comprising an elastomer matrix has a void space of, e.g., about 50% of the total volume of an elastomer matrix, about 60% of the total volume of an elastomer matrix, about 70% of the total volume of an elastomer matrix, about 80% of the total volume of an elastomer matrix, about 90% of the total volume of an elastomer matrix, about 95% of the total volume of an elastomer matrix, or about 97% of the total volume of an elastomer matrix. In other aspects of this embodiment, an open-cell surface foam material comprising an elastomer matrix has a void space of, e.g., at least 50% of the total volume of an elastomer matrix, at least 60% of the total volume of an elastomer matrix, at least 70% of the total volume of an elastomer matrix, at least 80% of the total volume of an elastomer matrix, at least 90% of the total volume of an elastomer matrix, at least 95% of the total volume of an elastomer matrix, or at least 97% of the total volume of an elastomer matrix. In yet other aspects of this embodiment, an open-cell surface foam material comprising an elastomer matrix has a void space of, e.g., at most 50% of the total volume of an elastomer matrix, at most 60% of the total volume of an elastomer matrix, at most 70% of the total volume of an elastomer matrix, at most 80% of the total volume of an elastomer matrix, at most 90% of the total volume of an elastomer matrix, at most 95% of the total volume of an elastomer matrix, or at most 97% of the total volume of an elastomer matrix. In yet other aspects of this embodiment, an open-cell surface foam material comprising an elastomer matrix has a void space of, e.g., about 50% to about 97% of the total volume of an elastomer matrix, about 60% to about 97% of the total volume of an elastomer matrix, about 70% to about 97% of the total volume of an elastomer matrix, about 80% to about 97% of the total volume of an elastomer matrix, about 90% to about 97% of the total volume of an elastomer matrix, about 50% to about 95% of the total volume of an elastomer matrix, about 60% to about 95% of the total volume of an elastomer matrix, about 70% to about 95% of the total volume of an elastomer matrix, about 80% to about 95% of the total volume of an elastomer matrix, about 90% to about 95% of the total volume of an elastomer matrix, about 50% to about 90% of the total volume of an elastomer matrix, about 60% to about 90% of the total volume of an elastomer matrix, about 70% to about 90% of the total volume of an elastomer matrix, or about 80% to about 90% of the total volume of an elastomer matrix.

The present specification discloses, in part, an open-cell surface foam material comprising an elastomer matrix defining an array of interconnected pores allowing substantial tissue growth into the interconnected pores in a time sufficient to reduce or prevent formation of fibrous capsules that can result in capsular contracture or scarring.

Thus, in an embodiment, an open-cell surface foam material comprising an elastomer matrix defining an array of interconnected pores allows tissue growth into the interconnected pores in a time sufficient to reduce or prevent formation of fibrous capsules that can result in capsular contracture or scarring. In aspects of this embodiment, an open-cell surface foam material comprising an elastomer matrix defining an array of interconnected pores allows tissue growth into the interconnected pores sufficient to reduce or prevent formation of fibrous capsules in, e.g., about 2 days after implantation, about 3 days after implantation, about 4 days after implantation, about 5 days after implantation, about 6 days after implantation, about 7 days, about 2 weeks after implantation, about 3 weeks after implantation, or about 4 weeks after implantation. In other aspects of this embodiment, an open-cell surface foam material comprising an elastomer matrix defining an array of interconnected pores allows tissue growth into the interconnected pores sufficient to reduce or prevent formation of fibrous capsules in, e.g., at least 2 days after implantation, at least 3 days after implantation, at least 4 days after implantation, at least 5 days after implantation, at least 6 days after implantation, at least 7 days, at least 2 weeks after implantation, at least 3 weeks after implantation, or at least 4 weeks after implantation. In yet other aspects of this embodiment, an open-cell surface foam material comprising an elastomer matrix defining an array of interconnected pores allows tissue growth into the interconnected pores sufficient to reduce or prevent formation of fibrous capsules in, e.g., at most 2 days after implantation, at most 3 days after implantation, at most 4 days after implantation, at most 5 days after implantation, at most 6 days after implantation, at most 7 days, at most 2 weeks after implantation, at most 3 weeks after implantation, or at most 4 weeks after implantation. In still other aspects of this embodiment, an open-cell surface foam material comprising an elastomer matrix defining an array of interconnected pores allows tissue growth into the interconnected pores sufficient to reduce or prevent formation of fibrous capsules in, e.g., about 2 days to about 4 days after implantation, about 2 days to about 5 days after implantation, about 2 days to about 6 days after implantation, about 2 days to about 7 days after implantation, about 1 week to about 2 weeks after implantation, about 1 week to about 3 weeks after implantation, or about 1 week to about 4 weeks after implantation.

An open-cell surface foam material comprising an elastomer matrix generally has a low level of microporosity. As used herein, the term “microporosity” refers to a measure of the presence of small micropores within an open-cell surface foam material comprising an elastomer matrix itself (as opposed to the pores defined by an elastomer matrix). In some embodiments, all or substantially all of the micropores in an elastomer matrix are between about 0.1 μm and about 5 μm, such as between about 0.1 μm and about 3 μm or between about 0.1 μm and about 2 μm. The term “low level of microporosity” means that micropores represent less than 2% of the volume of an open-cell surface foam material comprising an elastomer matrix, as measured by measuring the percentage void space in a cross-section through an elastomer matrix.

The shape, roundness, and diameter of pores, the connections between pores, the total volume of the foam material, the void volume, and the elastomer matrix volume can all be assessed using scanning electron microscopy.

The present specification discloses in part, methods of making a foam material disclosed in the present specification.

In one aspect, a method of making an open-cell surface foam material, the method comprising the steps of: a) preparing a foaming solution, the foaming solution comprising an elastomer base and a curing agent; b) adding the foaming solution to a mold assembly, the mold assemble comprising a component having a textured surface comprising an interconnected array of void space, wherein the foaming solution is added to the mold assembly in a manner sufficient to allow penetration of the foaming solution into the interconnected array of void space; c) treating the foaming solution to produce a foam material; and d) removing the mold assembly, wherein removal of the textured surface component produces a foam material having an open-cell surface.

Aspects of the present specification provide, in part, preparing a foaming solution. A foaming solution comprises an elastomer base and a curing agent. As used herein, the term “elastomer base” refers to an elastomer disclosed in the present specification in its uncured state. As used herein, the term “silicon-based elastomer base” refers to a silicon-based elastomer disclosed in the present specification in its uncured state. As used herein, the term “curing agent” refers to an agent that facilitates the curing of an elastomer. As used herein, the term “curing” refers to a process that exposes the chains of a elastomer to a element which activates a phase change in the elastomer to a more stable state, such as, e.g., by physically or chemically cross-linked elastomer chains to one another. Non-limiting examples of curing include thermal curing, chemical curing, catalyst curing, radiation curing, and physical curing. Curing of a foaming solution comprising an elastomer base and a curing agent can be done under any condition for any length of time with the proviso that the curing forms an foam material as disclosed in the present specification that is sufficient to allow tissue growth within its array of interconnected of pores. Non-limiting examples of curing agents used for catalyst curing include, without limitation, metal catalysts like platinum catalysts and tin catalysts, acetoxy catalysts, oxime catalysts, peroxide catalysts, and amine catalysts.

To prepare a foaming solution disclosed in the present specification, an elastomer base and a curing agent are mixed together in a set ratio that ensures proper aeration of the mixture before the mixture completely cures. This ratio is based on the specific elastomer base and curing agent used. For example, a RTV silicone and metal catalyst are generally mixed in a ratio of 100:6. In addition, the relative quantities in which the elastomer base and a curing agent are mixed may be varied in order to produce foams with different properties. For instance, using a greater quantity of the curing agent may cause the mixture to cure more quickly, leaving less time for the foaming process to settle. Intermediate conditions may also be altered in this manner. For instance, lowering the amount of curing agent used may increase the working time available in creating a foam. This mixture is then aerated by circulating air through the mixture, mixing the mixture with air, or dissolving air into the mixture to produce gas bubbles. Aeration of the elastomer-agent mixture can be accomplished by any suitable means that can pass the mixture through air or pass air through the mixture, including, without limitation, mechanical aeration such as, e.g., stirring, cascades, aeration turbines, compressed air, and/or any combination thereof. Parameters of an aeration process may be configured to produce a foam material with particularly desired properties. For example, the speed of agitation, the duration of aeration, the temperature used during aeration, the atmospheric pressure used during aeration, the mechanical device used to accomplish aeration, and/or any combination thereof can be adjusted to produce a foam material with the desired properties. For example, the density of a foam material can be lowered by increasing the speed of the agitation.

Other materials may also be added in an elastomer-agent mixture as well. For instance, materials which may contribute to the physical properties of the foam, such as, e.g. affecting the foams porosity, appearance, strength, flexibility, and/or any combination thereof. In one example, a foaming agent may be added that increases the rate of foaming or number of bubbles formed. In addition, using more or less of the foaming agent may generate a greater or lesser quantity of bubbles, determining the porosity of the resulting foam. As another example, a non-foaming material may be used. For instance, it may be desirable in some applications to form a foam material having solid portions and foam portions.

Aspects of the present specification provide, in part, adding the foaming solution into a mold assembly, where the mold assemble comprises a component having a textured surface.

A mold assembly may comprise any desired shape or configuration, have many different and complex surface geometries, and may comprise a number of components. For instance, a mold assembly may simply comprise a base component and a component having a textured surface, where the textured surface component covers all or a portion of the base component. Such mold assemblies are useful in producing flat sheets of foam material. The base can also be modified in that it may comprise a concave recess where a component of textured surface is located in the recess area. Such molds are useful in producing curved or rounded open cell surface foam materials where the open cell surface is on the concave surface of the foam. Alternatively, such foams could be inverted, thereby creating curved or rounded open cell surface foam materials where the open cell surface is on the convex surface of the foam. In this configuration, a component having a textured surface could be reversibly or irreversibly attached to the base, or an integral part of the base.

A mold assembly may comprise a plurality of component parts. In some examples, a two part mold assembly may be used, such as, e.g., a mold assembly having a bottom component and a top component. A mold assembly shaped as a container may also be used, such as, e.g., molds having a hollow internal recess into which a foaming solution is poured through an opening.

The component parts of a mold assembly may be made of any reasonable material, and a particular mold assembly used may reflect the shape of the foam object to be formed. For example, the mold assembly chosen may be generally in the shape of a foam material to be created, such as, e.g. having an internal cavity in which the foam solution may cure which may impose the general shape of the intended object on the curing foam.

Other characteristics of the mold may be chosen as well, such as, e.g., the material used to make the component parts, the size of the mold assembly and component parts, whether the mold is reusable, etc. These characteristics may be chosen to suit the intended application. For instance, molds of different materials may be used based on the size of the intended foam object, the complexity of the shape to be formed, the chemical composition of the foam material, the cleaning process to be used with the mold, etc.

Aspects of the present specification provide, in part, a mold assembly comprises a component having a textured surface. As used herein, the term “component having a textured surface” or “textured surface component” refers to a component material having a three-dimensional framework of material that forms an interconnected array of void space. The term “void space” when used in reference to a textured surface material refers to the actual or physical space in a textured surface material, i.e., the actual space not physically occupied by the material comprising the textured surface material. As such, the total volume of a textured surface material disclosed in the present specification is based upon the textured surface material space and the void space. The interconnected array of void space in textured surface material must be of sufficient depth so as to allow sufficient penetration or integration of the foaming solution into the void spaces of the textured surface material. As used herein, the term “sufficient penetration or integration” when in reference to a foaming solution refers to a penetration or integration of the foaming solution into the interconnected array of void space that, upon curing of the foaming solution, removal of the textured surface component produces a foam material having an open-cell surface. A component having a textured surface may be any material which will allow the foaming solution to penetrate or integrate into the void space of the textured component.

Void spaces of the textured material can have any shape with the proviso that the void space shape is sufficient to allow penetration or integration of the foaming solution into the interconnected array of void space as disclosed in the present specification. Useful void space shapes include, without limitation, roughly spherical, perfectly spherical, ellipsoidal, polyhedronal, triangular, pyramidal, quadrilateral like squares, rectangles, parallelograms, trapezoids, rhombus, kites, other types of polygonal shapes, cylindrical, conical, helical, braided, twisted, other types of tubular shapes, random geometries, and/or any combination thereof.

Thus, in an embodiment, a mold assembly comprises a textured surface component comprising an interconnected array of void space. In an aspect of this embodiment, a mold assembly comprises a textured surface component comprising an interconnected array of polygonal shaped void spaces. In aspects of this embodiment, a mold assembly comprises a textured surface component comprising an interconnected array of, e.g., roughly spherical shaped void spaces, perfectly spherical shaped void spaces, ellipsoidal shaped void spaces, polyhedronal shaped void spaces, triangular shaped void spaces, pyramidal shaped void spaces, quadrilateral like square shaped void spaces, rectangle shaped void spaces, parallelogram shaped void spaces, trapezoid shaped void spaces, rhombus shaped void spaces, kite shaped void spaces, or any combination thereof. In another aspect of this embodiment, a mold assembly comprises a textured surface component comprising an interconnected array of tubular shaped void spaces. In other aspects of this embodiment, a mold assembly comprises a textured surface component comprising an interconnected array of, e.g., cylindrical shaped void spaces, conical shaped void spaces, helical shaped void spaces, braided shaped void spaces, twisted shaped void spaces, or any combination thereof.

In another embodiment, a textured surface component comprising an interconnected array of void space has a void space sufficient to allow penetration or integration of the foaming solution into the textured component. In aspects of this embodiment, a textured surface component comprising an interconnected array of void space has a void space of, e.g., about 50% of the total volume of a textured surface component, about 60% of the total volume of a textured surface component, about 70% of the total volume of a textured surface component, about 80% of the total volume of a textured surface component, about 90% of the total volume of a textured surface component, about 95% of the total volume of a textured surface component, or about 97% of the total volume of a textured surface component. In other aspects of this embodiment, a textured surface component comprising an interconnected array of void space has a void space of, e.g., at least 50% of the total volume of a textured surface component, at least 60% of the total volume of a textured surface component, at least 70% of the total volume of a textured surface component, at least 80% of the total volume of a textured surface component, at least 90% of the total volume of a textured surface component, at least 95% of the total volume of a textured surface component, or at least 97% of the total volume of a textured surface component. In yet other aspects of this embodiment, a textured surface component comprising an interconnected array of void space has a void space of, e.g., at most 50% of the total volume of a textured surface component, at most 60% of the total volume of a textured surface component, at most 70% of the total volume of a textured surface component, at most 80% of the total volume of a textured surface component, at most 90% of the total volume of a textured surface component, at most 95% of the total volume of a textured surface component, or at most 97% of the total volume of a textured surface component. In yet other aspects of this embodiment, a textured surface component comprising an interconnected array of void space has a void space of, e.g., about 50% to about 97% of the total volume of a textured surface component, about 60% to about 97% of the total volume of a textured surface component, about 70% to about 97% of the total volume of a textured surface component, about 80% to about 97% of the total volume of a textured surface component, about 90% to about 97% of the total volume of a textured surface component, about 50% to about 95% of the total volume of a textured surface component, about 60% to about 95% of the total volume of a textured surface component, about 70% to about 95% of the total volume of a textured surface component, about 80% to about 95% of the total volume of a textured surface component, about 90% to about 95% of the total volume of a textured surface component, about 50% to about 90% of the total volume of a textured surface component, about 60% to about 90% of the total volume of a textured surface component, about 70% to about 90% of the total volume of a textured surface component, or about 80% to about 90% of the total volume of a textured surface component.

In another embodiment, a mold assembly comprises a textured surface component comprising an interconnected array of void space has a depth sufficient to allow penetration or integration of a foaming solution into the void space of the textured component. In an aspect of this embodiment, a textured surface component has a void space depth comprising the entire depth of a component. In other aspects of this embodiment, a textured surface component has a void space depth of, e.g., about 0.1 mm, about 0.25 mm, about 0.5 mm, about 0.75 mm, about 1 mm, about 2 mm, about 3 mm, about 4 mm, about 5 mm, about 6 mm, about 7 mm, about 8 mm, about 9 mm, or about 10 mm. In yet other aspects of this embodiment, a textured surface component has a void space depth of, e.g., at least 0.1 mm, at least 0.25 mm, at least 0.5 mm, at least 0.75 mm, at least 1 mm, at least 2 mm, at least 3 mm, at least 4 mm, at least 5 mm, at least 6 mm, at least 7 mm, at least 8 mm, at least 9 mm, or at least 10 mm. In still other aspects of this embodiment, a textured surface component has a void space depth of, e.g., at most 0.1 mm, at most 0.25 mm, at most 0.5 mm, at most 0.75 mm, at most 1 mm, at most 2 mm, at most 3 mm, at most 4 mm, at most 5 mm, at most 6 mm, at most 7 mm, at most 8 mm, at most 9 mm, or at most 10 mm. In further aspects of this embodiment, a component having a textured surface comprises a textured surface having a depth of about 0.1 mm to about 10 mm, about 0.25 mm to about 10 mm, about 0.5 mm to about 10 mm, about 0.75 mm to about 10 mm, about 1 mm to about 10 mm, about 2 mm to about 10 mm, about 3 mm to about 10 mm, about 4 mm to about 10 mm, or about 5 mm to about 10 mm, about 0.1 mm to about 5 mm, about 0.25 mm to about 5 mm, about 0.5 mm to about 5 mm, about 0.75 mm to about 5 mm, about 1 mm to about 5 mm, about 2 mm to about 5 mm, or about 3 mm to about 5 mm.

The properties of a textured surface component material may be chosen to suit the particular application, such as, e.g. the material of the textured surface component itself, the structure of the texture surface including porosity, interconnectivity of void spaces, and interconnection size of void spaces, the shape of the component, and/or any combination thereof. It is noted, that a textured surface component material may be matched to the foam material being created. As a non-limiting example, a textured surface component material may be chosen such that it remains significantly separate from a foam material, making it easy to remove the textured surface component from the foam material at the end of the process. As another non-limiting example, a foam material having an interconnected array of spherical pores may be match to a textured surface component having an interconnected array of spherical shaped void spaces. As another non-limiting example, a foam material having an interconnected array of tubular pores may be match to a textured surface component having an interconnected array of tubular shaped void spaces.

Together with the specifics of the mixing and treating processes the materials used in both the textured surface component and the foam material may help to determine the extent to which the foam material penetrates or integrates into the void space of the textured component. For example, the viscosity of the foaming mixture and the wet ability between the foam solution and the material of the component having a textured surface may affect the level of penetration or integration into the void space of the textured component. For instance greater penetration or integration into the void space of the textured surface component may occur if the materials are of high surface energies and therefore wet each other well. Greater penetration or integration into the void space of the textured component may also occur if the viscosity of the foaming solution is relatively low. As will be described below, greater penetration or integration into the void space of the textured component may lead to more certain skin removal, but may, however, complicate the removal process.

In addition, a textured surface component material may be chosen to have chemical or physical properties which may allow it to be easily removed from the foam material once the curing process is complete. For instance, a textured surface component material may be chosen to be soluble in one or more substances in which the foam will not be soluble. In addition, a textured surface component material may be chosen to withstand a cleaning process, such as, e.g. the application of heat, solvents, and/or any combination thereof.

A component having a textured surface may be integrated into another component part of a mold assembly. For example, a component part of a mold assembly can have its surface textured by sintering a compound to the surface, such as, e.g., a metal, a ceramic, a polymer, or any combination thereof. As another example, a component part of a mold assembly can have its surface textured by scoring or etching the surface. Such integration methods are known in the art and exemplar methods are described in, e.g., Brooke and Miller, Rapid Texture Prototyping; U.S. Pat. No. 6,558,496; Williamson, et al., Method of Making a Mold and Parts from the Mold, U.S. Pat. No. 5,580,507; Malkowski, Texturing a Mold Surface, U.S. Pat. No. 4,956,200; Summitt and Graham, Texturing of Molding Dies, U.S. 2009/0226619; and Popiolkowski, et al., Method of Surface Texturing, U.S. 2004/0056211; each of which is incorporated by reference in its entirety.

Alternatively, a component having a textured surface may be a separate component that is reversibly or irreversibly attached to another component part of a mold assembly. For example, a separate component part having a textured surface can be inserted into the mold assembly at a desired location. In this aspect, the material used for a textured surface of the component may be selected from any type of material comprising an interconnected array of void space into which the foaming solution may penetrate or integrate. For example, a textured surface component having a textured surface may be a foam, such as, e.g., an elastomer-based foam, a ceramic-based foam, a metal-composite-based foam, or any combination thereof. As another example, a textured surface component having a textured surface may be in the form of a felt, a mesh, a screen, or any other type of structure where threads, fibers, or filaments of a material may be woven, bundled or twisted together. Non-limiting examples of a separate textured surface component include a polymer such as polypropylene, polyurethane, silicone, polyolefin, melamine, cellulose, as well as any other suitable material such as a ceramic, a metal, a composite material, a natural fiber, a stone, a glass, and/or any combination thereof. Ceramic oxides include, without limitation, alumina and zirconia. Ceramic non-oxides include, without limitation, carbides, borides, nitrides, and silicides. Metals include, without limitation, aluminum, steel, titanium, iron, cobalt, nickel, copper, tin, gold, silver, bronze, and magnesium. Composite materials include, without limitation, fiber reinforced polymers, carbon reinforced plastics, glass reinforced plastics, thermoplastic composites, short fiber thermoplastics, long fiber, thermoplastics, long fiber-reinforced thermoplastics, ceramic particulate reinforced composites, and combinations of ceramic oxides and ceramic non-oxides. Fibers include, without limitation, cellulose fibers like cotton, jute, flax, ramie, sisal, and hemp. Fibers containing collagen and keratin like silk, sinew, wool, catgut, angora, mohair, and alpaca. Stones include, without limitation igneous, sedimentary, and metamorphic. Glass includes, without limitation, silica glass, and non-silica glass.

A mold assembly comprising a component having a textured surface may comprise a single textured surface component. See, e.g., FIG. 1B. In aspects where a component having a textured material is a separate component, a textured surface component may simply be placed unsecured in or on another component of a mold assembly. In other aspects where a textured surface component is attached to another component of a mold assembly, attachment may be accomplished using any suitable method, such as, e.g. using an adhesive, using a vacuum, using heat, using hardware such as screws, pins, or clips. Attachment of an textured surface component can be reversible in that the textured surface component can be detached from the other component of the mold assembly. Attachment of a textured surface component can be irreversible in that the textured surface component is permanently affixed to another component of a mold assembly, such as, e.g., by sintering, welding, and/or any combination thereof. In addition, a textured surface component may be attached to any portion of another component of the mold assembly as desired. For instance using a two part mold assembly, a textured surface component may be attached to both component parts, or to only one of the two component parts.

A mold assembly comprising a component having a textured surface may comprise a plurality of textured surface components. For example, in a two-part mold assembly, a first component of the mold assembly comprises a first component having a textured surface and a second component of the mold assembly comprises a second component having a textured surface. See, e.g., FIG. 1C. In this case, a first component of the mold assembly comprises a first textured surface component is lowered to cover a second component of the mold assembly comprising a second textured surface component. The foaming solution may already be added to the second component part or may be added after the first and second component parts of the mold assembly are in place. The end result is a foam material having two open cell surfaces.

In addition, a component having a textured surface need not be placed over the entire area on which the foaming solution is to be disposed. For instance, a textured surface component may be placed on only a portion of another component part of the mold assembly. In such a way, an open cell surface foam material may be created which may have an open cell surface in one area, while having a non-porous or less-porous surface in other area.

Aspects of the present specification provide, in part, a foam solution is added to a mold assembly. Adding a foam solution to a mold assembly can be accomplished using any suitable method with the proviso that the foaming solution penetrates or integrates into the void space of the textured surface component in a manner sufficient to allow removal of a surface of foam material thereby making an open-cell foam material disclosed in the present specification. Non-limiting examples of adding a foam solution to a mold assembly include pouring, injecting, molding, dipping, casting, curtain coating, brushing, scooping, spraying, and/or any combination thereof. The foaming solution may be added to the mold assembly while it is still in an uncured state, or only partially cured, and the solution may be actively generating bubbles which will create a matrix defining an array of interconnected pores. In addition, the foam solution need not be disposed uniformly. Rather, the foam solution may be applied with different thicknesses on different portions of the mold assembly, at different times (e.g. in layers). Further, more than one foaming solution may be used, such as, e.g. on different portions of the mold assembly or in different layers.

The time and force with which these additional textured layers are pressed into the foaming solution may be varied depending on the application. For example, additional textured layers may be pressed onto a foaming solution immediately, while in other cases, the foaming solution may be allowed to partially cure before a textured surface is pressed into the additional layer of foaming solution. In addition, a textured surface may be pressed into the foaming solution at a predetermined depth and/or with a predetermined force, or the textured surface may simply be allowed to settle on the foaming solution. In addition, such additional components having a textured surface need not be attached to another component of a mold assembly. Rather, a component having a textured surface may be applied directly to the foaming solution. For example, a sheet comprising a component having a textured surface may be placed on top of a foaming solution, e.g. being placed on top of an open mold. Similarly, the foaming solution may be wrapped in a component having a textured surface.

Aspects of the present specification provide, in part, treating the foaming solution to produce a foam material comprising an elastomer matrix defining an array of interconnected pores. As used herein, the term “treating” refers to a process that cures the elastomer base to form an elastomer matrix defining an array of interconnected of pores as disclosed in the present specification. As used herein, the term “curing” refers to a process that exposes the chains of a polymer to a element which activates a phase change in the polymer to a more stable state, such as, e.g., by physically or chemically cross-linked polymer chains to one another. Non-limiting examples of treating include temporal treating, thermal treating, chemical treating, catalyst treating, radiation treating, and physical treating. Treatment of a foaming solution can be accomplished under any condition for any length of time with the proviso that the treating cures the elastomer base to form an elastomer matrix defining an array of interconnected of pores as disclosed in the present specification. For example, a foaming solution may be treated simply by allowing the solution to stand at an ambient temperature of about 18° C. to about 22° C. In other aspects, treatment of the foam solution may be facilitated by the application of heat or pressure. Depending on the application, a foaming solution may be allowed to completely cure or may be allowed to only partially cure before removal from the mold assembly.

In aspects of this embodiment, a treating the foaming solution produces a substantially non-degradable elastomer matrix defining an array of interconnected pores, a biocompatible elastomer matrix defining an array of interconnected pores, or a substantially non-degradable, biocompatible elastomer matrix defining an array of interconnected pores.

Aspects of the present specification provide, in part, removing a mold assembly, wherein removal of a component having a textured surface produces a foam material having an open-cell surface. Removal of a textured surface component can be accomplished by any method that produces a foam material having an open-cell surface including, without limitation, physical removal, solvent removal, thermal removal, and/or any combination thereof.

In one aspect, removal is accomplished by physical removal. This removal method typically uses either 1) a mold assembly comprising a separate component having a textured surface and another component part, where the textured surface component may or may not be detached from the other component; or 2) a mold assembly comprising a component having a textured surface and another component part, where the textured surface component is irreversible affixed to the other component of the mold assembly. In a physical removal method, the mold assembly is dismantled in such a manner that the foam material is delaminated by a textured surface component as the component is removed from the cured foam material, thereby producing a foam material having an open-cell surface. As used herein, the term “delaminate” refers to the removal of the surface portion of the cured foam material at or near the site of void space penetration or integration with the textured surface of the component to create a surface having an open cell structure. In essence, removal of the textured surface component tears the foam material at or near the site where the foam has penetrated or integrated into the void space of the textured component.

In another aspect, removal is accomplished by solvent removal. This removal method typically uses a mold assembly comprising a separate component having a textured surface and another component part, where the textured surface component is detachable from the other component a mold assembly. In a solvent removal method, the mold assembly is dismantled in such a manner that leaves a textured surface component in contact with the foam material. This foam material-component piece in then placed in a solution for which the textured surface component is solvent, but the foam material in insolvent or substantially insolvent. Under the appropriate conditions and time, the solvent will dissolve the textured surface component leaving behind a foam material with an open-cell surface. A solvent removal method can incorporate a plurality of solution changes over time to facilitate removal of the textured surface component. Non-limiting examples of solvents useful in a solvent removal method include water, methylene chloride, acetic acid, formic acid, pyridine, tetrahydrofuran, dimethylsulfoxide, dioxane, benzene, and/or mixtures thereof. A mixed solvent can be in a ratio of higher than about 1:1, first solvent to second solvent or lower than about 1:1, first solvent to second solvent.

In yet another aspect, removal is accomplished by thermal removal. This removal method typically uses either 1) a mold assembly comprising a separate component having a textured surface and another component part, where the textured surface component may or may not be detached from the other component; or 2) a mold assembly comprising a component having a textured surface and another component part, where the textured surface component is irreversible affixed to the other component of the mold assembly. In a thermal removal method, the mold assembly, which may or may not be dismantled, is heated in a manner that degrades or burns off a textured surface component, but does not adversely affect the foam material. Under the appropriate conditions and time, the heat treatment will remove the textured surface component leaving behind a foam material with an open-cell surface.

In still another aspect, removal is accomplished by degradation removal. This removal method typically uses a mold assembly comprising a separate component having a textured surface and another component, where the textured surface component may be detached from the other component. In a degradation removal method, the mold assembly is dismantled in such a manner that leaves a textured surface component in contact with the foam material. This foam material-component piece in then placed in a solution comprising a compound or compounds that degrade the textured surface component, but for which the foam material remains intact or substantially intact. Under the appropriate conditions and time, the compound solution will destroy the textured surface component leaving behind a foam material with an open-cell surface. A degradation removal method can incorporate a plurality of compound solution changes over time to facilitate removal of the textured surface component. Non-limiting examples of compounds useful in a degradation removal method include water, radiation, ultraviolet light, enzymes, and/or mixtures thereof. A mixed compound solution can be in a ratio of higher than about 1:1, first compound to second compound or lower than about 1:1, first compound to second compound.

Thus, in an embodiment, a mold assembly comprising a textured surface component is removed from a foam material, wherein removal produces a foam material having an open-cell surface. In aspects of this embodiment, a mold assembly comprising a textured surface component is removed from a foam material, where the removal removes, e.g., about 75% of the textured surface component, about 80% of the textured surface component, about 85% of the textured surface component, about 90% of the textured surface component, or about 95% of the textured surface component. In other aspects of this embodiment, a mold assembly comprising a textured surface component is removed from a foam material, where the removal removes, e.g., at least 75% of the textured surface, at least 80% of the textured surface component, at least 85% of the textured surface component, at least 90% of the textured surface component, or at least 95% of the textured surface component. In aspects of this embodiment, a mold assembly comprising a textured surface component is removed from a foam material, where the removal removes, e.g., about 75% to about 90% of the textured surface component, about 75% to about 95% of the textured surface component, about 75% to about 100% of the textured surface component, about 80% to about 90% of the textured surface component, about 80% to about 95% of the textured surface component, about 80% to about 100% of the textured surface component, about 85% to about 90% of the textured surface component, about 85% to about 95% of the textured surface component, about 85% to about 100% of the textured surface component, about 90% to about 100% of the textured surface component, or about 95% to about 100% of the textured surface component. In an aspect, a mold assembly comprising a textured surface component is removed from a foam material by, e.g., a physical removal method, a solvent removal method, a thermal removal method, a degradation removal method, and/or any combination thereof.

In another embodiment, a mold assembly comprising a textured surface component is removed from a foam material by a physical removal method, wherein removal produces a foam material having an open-cell surface. In aspects of this embodiment, a mold assembly comprising a textured surface component is removed from a foam material by a physical removal method, where the removal removes, e.g., about 75% of the textured surface component, about 80% of the textured surface component, about 85% of the textured surface component, about 90% of the textured surface component, or about 95% of the textured surface component. In other aspects of this embodiment, a mold assembly comprising a textured surface component is removed from a foam material by a physical removal method, where the removal removes, e.g., at least 75% of the textured surface component, at least 80% of the textured surface component, at least 85% of the textured surface component, at least 90% of the textured surface component, or at least 95% of the textured surface component. In aspects of this embodiment, a mold assembly comprising a textured surface component is removed from a foam material by a physical removal method, where the removal removes, e.g., about 75% to about 90% of the textured surface component, about 75% to about 95% of the textured surface component, about 75% to about 100% of the textured surface component, about 80% to about 90% of the textured surface component, about 80% to about 95% of the textured surface component, about 80% to about 100% of the textured surface component, about 85% to about 90% of the textured surface component, about 85% to about 95% of the textured surface component, about 85% to about 100% of the textured surface component, about 90% to about 100% of the textured surface component, or about 95% to about 100% of the textured surface component.

In yet another embodiment, a mold assembly comprising a textured surface component is removed from a foam material by a solvent removal method, wherein removal produces a foam material having an open-cell surface. In aspects of this embodiment, a mold assembly comprising a textured surface component is removed from a foam material by a solvent removal method, where the removal removes, e.g., about 75% of the textured surface component, about 80% of the textured surface component, about 85% of the textured surface component, about 90% of the textured surface component, or about 95% of the textured surface component. In other aspects of this embodiment, a mold assembly comprising a textured surface component is removed from a foam material by a solvent removal method, where the removal removes, e.g., at least 75% of the textured surface component, at least 80% of the textured surface component, at least 85% of the textured surface component, at least 90% of the textured surface component, or at least 95% of the textured surface component. In aspects of this embodiment, a mold assembly comprising a textured surface component is removed from a foam material by a solvent removal method, where the removal removes, e.g., about 75% to about 90% of the textured surface component, about 75% to about 95% of the textured surface component, about 75% to about 100% of the textured surface component, about 80% to about 90% of the textured surface component, about 80% to about 95% of the textured surface component, about 80% to about 100% of the textured surface component, about 85% to about 90% of the textured surface component, about 85% to about 95% of the textured surface component, about 85% to about 100% of the textured surface component, about 90% to about 100% of the textured surface component, or about 95% to about 100% of the textured surface component.

In yet another embodiment, a mold assembly comprising a textured surface component is removed from a foam material by a thermal removal method, wherein removal produces a foam material having an open-cell surface. In aspects of this embodiment, a mold assembly comprising a textured surface component is removed from a foam material by a thermal removal method, where the removal removes, e.g., about 75% of the textured surface component, about 80% of the textured surface component, about 85% of the textured surface component, about 90% of the textured surface component, or about 95% of the textured surface component. In other aspects of this embodiment, a mold assembly comprising a textured surface component is removed from a foam material by a thermal removal method, where the removal removes, e.g., at least 75% of the textured surface component, at least 80% of the textured surface component, at least 85% of the textured surface component, at least 90% of the textured surface component, or at least 95% of the textured surface component. In aspects of this embodiment, a mold assembly comprising a textured surface component is removed from a foam material by a thermal removal method, where the removal removes, e.g., about 75% to about 90% of the textured surface component, about 75% to about 95% of the textured surface component, about 75% to about 100% of the textured surface component, about 80% to about 90% of the textured surface component, about 80% to about 95% of the textured surface component, about 80% to about 100% of the textured surface component, about 85% to about 90% of the textured surface component, about 85% to about 95% of the textured surface component, about 85% to about 100% of the textured surface component, about 90% to about 100% of the textured surface component, or about 95% to about 100% of the textured surface component.

In still another embodiment, a mold assembly comprising a textured surface component is removed from a foam material by a degradation removal method, wherein removal produces a foam material having an open-cell surface. In aspects of this embodiment, a mold assembly comprising a textured surface component is removed from a foam material by a degradation removal method, where the removal removes, e.g., about 75% of the textured surface component, about 80% of the textured surface component, about 85% of the textured surface component, about 90% of the textured surface component, or about 95% of the textured surface component. In other aspects of this embodiment, a mold assembly comprising a textured surface component is removed from a foam material by a degradation removal method, where the removal removes, e.g., at least 75% of the textured surface component, at least 80% of the textured surface component, at least 85% of the textured surface component, at least 90% of the textured surface component, or at least 95% of the textured surface component. In aspects of this embodiment, a mold assembly comprising a textured surface component is removed from a foam material by a degradation removal method, where the removal removes, e.g., about 75% to about 90% of the textured surface component, about 75% to about 95% of the textured surface component, about 75% to about 100% of the textured surface component, about 80% to about 90% of the textured surface component, about 80% to about 95% of the textured surface component, about 80% to about 100% of the textured surface component, about 85% to about 90% of the textured surface component, about 85% to about 95% of the textured surface component, about 85% to about 100% of the textured surface component, about 90% to about 100% of the textured surface component, or about 95% to about 100% of the textured surface component.

Depending on the removal method, once the foam material is removed from a component having a textured surface, the textured surface component and/or the other components of the mold assembly may be prepared for reuse. Any technique may be used to remove the remaining foam material that is suitable given the composition of the foam and the composition of the textured surface component and/or the other components of the mold assembly. For instance, if a textured surface component was not destroyed, the foam material remaining in the textured surface component may be removed from the textured surface by, e.g., dissolving, heating, degrading, washing, and/or any combination thereof. Once the foam material is removed, a textured surface component and/or the other components of the mold assembly may be reused to create another foam material.

The present specification discloses in part, biocompatible implantable device comprising a layer of foam material as disclosed in the present specification, wherein the foam material covers a surface of the device. See, e.g., FIG. 2, FIGS. 4-8. As used herein, the term “implantable” refers to any material that can be embedded into, or attached to, tissue, muscle, organ or any other part of an animal body. As used herein, the term “animal” includes all mammals including a human. A biocompatible implantable device is synonymous with “medical device”, “biomedical device”, “implantable medical device” or “implantable biomedical device” and includes, without limitation, pacemakers, dura matter substitutes, implantable cardiac defibrillators, tissue expanders, and tissue implants used for prosthetic, reconstructive, or aesthetic purposes, like breast implants, muscle implants or implants that reduce or prevent scarring. Examples of biocompatible implantable devices that the foam material disclosed in the present specification can be attached to are described in, e.g., Schuessler, Rotational Molding System for Medical Articles, U.S. Pat. No. 7,628,604; Smith, Mastopexy Stabilization Apparatus and Method, U.S. Pat. No. 7,081,135; Knisley, Inflatable Prosthetic Device, U.S. Pat. No. 6,936,068; Falcon, Reinforced Radius Mammary Prostheses and Soft Tissue Expanders, U.S. Pat. No. 6,605,116; Schuessler, Rotational Molding of Medical Articles, U.S. Pat. No. 6,602,452; Murphy, Seamless Breast Prosthesis, U.S. Pat. No. 6,074,421; Knowlton, Segmental Breast Expander For Use in Breast Reconstruction, U.S. Pat. No. 6,071,309; VanBeek, Mechanical Tissue Expander, U.S. Pat. No. 5,882,353; Hunter, Soft Tissue Implants and Anti-Scarring Agents, Schuessler, Self-Sealing Shell For Inflatable Prostheses, U.S. Patent Publication 2010/0049317; U.S. 2009/0214652; Schraga, Medical Implant Containing Detection Enhancing Agent and Method For Detecting Content Leakage, U.S. Patent Publication 2009/0157180; Schuessler, All-Barrier Elastomeric Gel-Filled Breast Prosthesis, U.S. Patent Publication 2009/0030515; Connell, Differential Tissue Expander Implant, U.S. Patent Publication 2007/0233273; and Hunter, Medical implants and Anti-Scarring Agents, U.S. Patent Publication 2006/0147492; Van Epps, Soft Filled Prosthesis Shell with Discrete Fixation Surfaces, International Patent Publication WO/2010/019761; Schuessler, Self Sealing Shell for Inflatable Prosthesis, International Patent Publication WO/2010/022130; Yacoub, Prosthesis Implant Shell, International Application No. PCT/US09/61045, each of which is hereby incorporated by reference in its entirety.

A biocompatible implantable device disclosed in the present specification can be implanted into the soft tissue of an animal during the normal operation of the device. Such implantable devices may be completely implanted into the soft tissue of an animal body (i.e., the entire device is implanted within the body), or the device may be partially implanted into an animal body (i.e., only part of the device is implanted within an animal body, the remainder of the device being located outside of the animal body). A biocompatible implantable device disclosed in the present specification can also be affixed to soft tissue of an animal during the normal operation of the medical device. Such devices are typically affixed to the skin of an animal body.

The present specification discloses, in part, an open cell surface foam material that covers a surface of the biocompatible implantable device. Any of the foam materials disclosed in the present specification can be used as the porous material covering a surface of a biocompatible implantable device. In general, the surface of a biocompatible implantable device is one exposed to the surrounding tissue of an animal in a manner that promotes tissue growth, and/or reduces or prevents formation of fibrous capsules that can result in capsular contracture or scarring.

Thus, in an embodiment, an open cell surface foam material covers the entire surface of a biocompatible implantable device. In another embodiment, an open cell surface foam material covers a portion of a surface of a biocompatible implantable device. In aspects of this embodiment, an open cell surface foam material covers to a front surface of a biocompatible implantable device or a back surface of a biocompatible implantable device. In other aspects, an open cell surface foam material covers only to, e.g., about 20%, about 30%, about 40%, about 50%, about 60%, about 70% about 80% or about 90% of the entire surface of a biocompatible implantable device, a front surface of a biocompatible implantable device, or a back surface of a biocompatible implantable device. In yet other aspects, an open cell surface foam material is applied only to, e.g., at least 20%, at least 30%, at least 40%, at least 50%, at least 60%, at least 70% at least 80% or at least 90% of the entire surface of a biocompatible implantable device, a front surface of a biocompatible implantable device, or a back surface of a biocompatible implantable device. In still other aspects, an open cell surface foam material is applied only to, e.g., at most 20%, at most 30%, at most 40%, at most 50%, at most 60%, at most 70% at most 80% or at most 90% of the entire surface of a biocompatible implantable device, a front surface of a biocompatible implantable device, or a back surface of a biocompatible implantable device. In further aspects, a porous material is applied only to, e.g., about 20% to about 100%, about 30% to about 100%, about 40% to about 100%, about 50% to about 100%, about 60% to about 100%, about 70% to about 100%, about 80% to about 100%, or about 90% to about 100% of the entire surface of a biocompatible implantable device, a front surface of a biocompatible implantable device, or a back surface of a biocompatible implantable device.

The layer of an open cell surface foam material covering a biocompatible implantable device can be of any thickness with the proviso that the material thickness allows tissue growth within the array of interconnected of pores of an elastomer matrix in a manner sufficient to reduce or prevent formation of fibrous capsules that can result in capsular contracture or scarring.

Thus, in an embodiment, a layer of an open cell surface foam material covering a biocompatible implantable device is of a thickness that allows tissue growth within the array of interconnected of pores of an elastomer matrix in a manner sufficient to reduce or prevent formation of fibrous capsules that can result in capsular contracture or scarring. In aspects of this embodiment, a layer of an open cell surface foam material covering a biocompatible implantable device comprises a thickness of, e.g., about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 10 mm. In other aspects of this embodiment, a layer an open cell surface foam material covering a biocompatible implantable device comprises a thickness of, e.g., at least 100 μm, at least 200 μm, at least 300 μm, at least 400 μm, at least 500 μm, at least 600 μm, at least 700 μm, at least 800 μm, at least 900 μm, at least 1 mm, at least 2 mm, at least 3 mm, at least 4 mm, at least 5 mm, at least 6 mm, at least 7 mm, at least 8 mm, at least 9 mm, or at least 10 mm. In yet other aspects of this embodiment, a layer of an open cell surface foam material covering a biocompatible implantable device comprises a thickness of, e.g., at most 100 μm, at most 200 μm, at most 300 μm, at most 400 μm, at most 500 μm, at most 600 μm, at most 700 μm, at most 800 μm, at most 900 μm, at most 1 mm, at most 2 mm, at most 3 mm, at most 4 mm, at most 5 mm, at most 6 mm, at most 7 mm, at most 8 mm, at most 9 mm, or at most 10 mm. In still other aspects of this embodiment, a layer of an open cell surface foam material covering a biocompatible implantable device comprises a thickness of, e.g., about 100 μm to about 500 μm, about 100 μm to about 1 mm, about 100 μm to about 5 mm, about 500 μm to about 1 mm, about 500 μm to about 2 mm, about 500 μm to about 3 mm, about 500 μm to about 4 mm, about 500 μm to about 5 mm, about 1 mm to about 2 mm, about 1 mm to about 3 mm, about 1 mm to about 4 mm, about 1 mm to about 5 mm, or about 1.5 mm to about 3.5 mm.

The present specification discloses in part, a method for making biocompatible implantable device comprising an open cell surface foam material. In an aspect, a method for making biocompatible implantable device comprises the step of attaching an open cell surface foam material to the surface of a biocompatible implantable device. In another aspect, a method for making biocompatible implantable device comprises the steps of a) preparing a surface of a biocompatible implantable device to receive an open cell surface foam material; b) attaching an open cell surface foam material to the prepared surface of the device. Any of the open cell surface foam materials disclosed in the present specification can be used as an open cell surface foam material attached to a surface of a biocompatible implantable device.

The present specification discloses, in part, preparing a surface of a biocompatible implantable device to receive an open cell surface foam material. Preparing such a surface of a biocompatible implantable device can be accomplished by any technique that does not destroy the desired properties of an open cell surface foam material or the biocompatible implantable device. As a non-limiting example, a surface of a biocompatible implantable device can be prepared by applying a bonding substance. Non-limiting examples of bonding substances include silicone adhesives, such as, e.g., RTV silicone and HTV silicone. The bonding substance is applied to the surface of a biocompatible implantable device, the open cell surface foam material, or both, using any method known in the art, such as, e.g., cast coating, spray coating, dip coating, curtain coating, knife coating, brush coating, vapor deposition coating, and the like.

The present specification discloses, in part, attaching an open cell surface foam material to a surface of a biocompatible implantable device. An open cell surface foam material can be attached to the entire surface of the device, or only to portions of the surface of the device. As a non-limiting example, an open cell surface foam material is attached only to the front surface of the device or only the back surface of the device. Attachment of an open cell surface foam material to a surface of a biocompatible implantable device can be accomplished by any technique that does not destroy the desired properties of the porous material or the biocompatible implantable device.

For example, attachment can occur by adhering an already formed open cell surface foam material onto a surface of a biocompatible implantable device using methods known in the art, such as, e.g., gluing, bonding, melting. For instance, a dispersion of silicone is applied as an adhesive onto a surface of a biocompatible implantable device, an open cell surface foam material sheet, or both, and then the two materials are placed together in a manner that allows the adhesive to attached the foam material to the surface of the device in such a way that there are no wrinkles on the surface of the device. The silicone adhesive is allowed to cure and then the excess material is cut off creating a uniform seam around the device. This process results in a biocompatible implantable device comprising an open cell surface foam material disclosed in the present specification. Examples 3 and 6 illustrate method of this type of attachment.

Regardless of the method of attachment, an open cell surface foam material can be applied to the entire surface of a biocompatible implantable device, or only to portions of the surface of a biocompatible implantable device. As a non-limiting example, an open cell surface foam material is applied only to the front surface of a biocompatible implantable device or only the back surface of a biocompatible implantable device.

Thus, in an embodiment, an open cell surface foam material is attached to a surface of a biocompatible implantable device by bonding a porous material to a surface of a biocompatible implantable device. In aspects of this embodiment, an open cell surface foam material is attached to a surface of a biocompatible implantable device by gluing, bonding, or melting the porous material to a surface of a biocompatible implantable device.

In another embodiment, an open cell surface foam material is applied to the entire surface of a biocompatible implantable device. In another embodiment, an open cell surface foam material is applied to a portion of a surface of a biocompatible implantable device. In aspects of this embodiment, an open cell surface foam material is applied to a front surface of a biocompatible implantable device or a back surface of a biocompatible implantable device. In other aspects, an open cell surface foam material is applied only to, e.g., about 20%, about 30%, about 40%, about 50%, about 60%, about 70% about 80% or about 90% of the entire surface of a biocompatible implantable device, a front surface of a biocompatible implantable device, or a back surface of a biocompatible implantable device. In yet other aspects, an open cell surface foam material is applied only to, e.g., at least 20%, at least 30%, at least 40%, at least 50%, at least 60%, at least 70% at least 80% or at least 90% of the entire surface of a biocompatible implantable device, a front surface of a biocompatible implantable device, or a back surface of a biocompatible implantable device. In still other aspects, an open cell surface foam material is applied only to, e.g., at most 20%, at most 30%, at most 40%, at most 50%, at most 60%, at most 70% at most 80% or at most 90% of the entire surface of a biocompatible implantable device, a front surface of a biocompatible implantable device, or a back surface of a biocompatible implantable device. In further aspects, a porous material is applied only to, e.g., about 20% to about 100%, about 30% to about 100%, about 40% to about 100%, about 50% to about 100%, about 60% to about 100%, about 70% to about 100%, about 80% to about 100%, or about 90% to about 100% of the entire surface of a biocompatible implantable device, a front surface of a biocompatible implantable device, or a back surface of a biocompatible implantable device.

The layer of an open cell surface foam material applied to a biocompatible implantable device can be of any thickness with the proviso that the material thickness allows tissue growth within the array of interconnected of pores of an elastomer matrix in a manner sufficient to reduce or prevent formation of fibrous capsules that can result in capsular contracture or scarring.

Thus, in an embodiment, a layer of an open cell surface foam material applied to a biocompatible implantable device is of a thickness that allows tissue growth within the array of interconnected of pores of an elastomer matrix in a manner sufficient to reduce or prevent formation of fibrous capsules that can result in capsular contracture or scarring. In aspects of this embodiment, a layer an open cell surface foam material applied to a biocompatible implantable device comprises a thickness of, e.g., about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 10 mm. In other aspects of this embodiment, a layer porous material applied to a biocompatible implantable device comprises a thickness of, e.g., at least 100 μm, at least 200 μm, at least 300 μm, at least 400 μm, at least 500 μm, at least 600 μm, at least 700 μm, at least 800 μm, at least 900 μm, at least 1 mm, at least 2 mm, at least 3 mm, at least 4 mm, at least 5 mm, at least 6 mm, at least 7 mm, at least 8 mm, at least 9 mm, or at least 10 mm. In yet other aspects of this embodiment, a layer an open cell surface foam material applied to a biocompatible implantable device comprises a thickness of, e.g., at most 100 μm, at most 200 μm, at most 300 μm, at most 400 μm, at most 500 μm, at most 600 μm, at most 700 μm, at most 800 μm, at most 900 μm, at most 1 mm, at most 2 mm, at most 3 mm, at most 4 mm, at most 5 mm, at most 6 mm, at most 7 mm, at most 8 mm, at most 9 mm, or at most 10 mm. In still other aspects of this embodiment, a layer an open cell surface foam material applied to a biocompatible implantable device comprises a thickness of, e.g., about 100 μm to about 500 μm, about 100 μm to about 1 mm, about 100 μm to about 5 mm, about 500 μm to about 1 mm, about 500 μm to about 2 mm, about 500 μm to about 3 mm, about 500 μm to about 4 mm, about 500 μm to about 5 mm, about 1 mm to about 2 mm, about 1 mm to about 3 mm, about 1 mm to about 4 mm, about 1 mm to about 5 mm, or about 1.5 mm to about 3.5 mm.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to foam materials, methods of forming such foam materials, biocompatible implantable devices comprising such foam materials, and methods of making such biocompatible implantable devices.

### Example 1

### A Method of Making an Open Cell Surface Foam Material Sheet

This example illustrates how to make a sheet of open cell surface foam material as disclosed in the present specification.

To prepare a mold assembly, a 20 cm×20 cm square base comprising a flat surfaced bottom and 2 cm ends around the perimeter of the base is prepared by reversibly attaching 20 cm×20 cm×5 mm polyurethane foam sheet to the base bottom with an adhesive. Alternatively, the polyurethane foam may be simply put atop the base bottom without attachment.

To prepare a foaming solution, 6 mL of a tin catalyst is added to 100 mL of RTV silicone base and the solution is aerated by vigorously mixing the solution for 30 seconds. A two-part RTV silicone foam, R-2370 is prepared by mixing 100 mL of R-2370 base with 6 mL of R-2370 catalyst. The sample is placed in a homogenizer with a propeller attachment and vigorously stirred at 50 rpm for 30 seconds at which point the viscosity is approximately 4,500 mPas.

The silicone foaming solution is then added to the mold assembly in a manner that allows penetration or integration of the solution into the void space of the polyurethane foam. The silicone foaming solution is then treated by allowing the solution to stand at ambient temperature until the foaming solution cured.

The mold assembly is then dismantled to remove the silicone foam material from the assembly. During this dismantling procedure, the polyurethane foam physically removes a surface portion of the cured silicone foam material at or near the site of penetration or integration into the void spaces to create a silicone foam material having an open cell surface. The resulting removal of the polyurethane foam produces a 20 cm×20 cm×5 mm silicone foam sheet having a first 20 cm×20 cm surface comprising an open cell structure and a second 20 cm×20 cm surface comprising a closed-cell surface. The approximate foam density is 0.16 g/cm³.

Alternatively, the mold assembly may be dismantled in such a manner that leaves the polyurethane foam in contact with the silicone foam. This silicone-polyurethane piece is then soaked in concentrated hydrochloric acid for a sufficient amount of time to allow the acid to dissolve the polyurethane foam. The resulting open cell surface silicone foam is then washed in deionized water. The resulting removal of the polyurethane foam produces a 20 cm×20 cm×5 mm silicone foam sheet having a first 20 cm×20 cm surface comprising an open cell structure and a second 20 cm×20 cm surface comprising a closed-cell surface.

A sample from the sheet of silicone foam material can be characterized by microCT analysis and/or scanning electron microscopy (SEM).

### Example 2

### A Method of Making an Open Cell Surface Foam Material Sheet This example illustrates how to make a sheet of open cell surface foam material as disclosed in the present specification.

A mold assembly is prepared as described in Example 1.

A foaming solution is prepared as described in Example 1.

The silicone foaming solution is then added to the mold assembly in a manner that allows penetration or integration of the solution into the void space of the polyurethane foam. A second 20 cm×20 cm×5 mm polyurethane foam sheet is then place on top of the foaming solution in a manner that allows penetration or integration of the solution into the void space of the second sheet of polyurethane foam. The silicone foaming solution is then treated by allowing the solution to stand at ambient temperature until the foaming solution cured.

The mold assembly is then dismantled to remove the silicone foam material from the assembly. During this dismantling procedure, both sheets of polyurethane foam physically remove a surface portion of the cured silicone foam material at or near the site of penetration or integration into the void spaces to create a silicone foam material having an open cell surface on both sides. The resulting removal of the polyurethane foam produces a 20 cm×20 cm×5 mm silicone foam sheet having a first 20 cm×20 cm surface comprising an open cell structure and a second 20 cm×20 cm surface comprising an open-cell surface.

Alternatively, the mold assembly may be dismantled in such a manner that leaves both polyurethane foam sheets in contact with the silicone foam. This silicone-polyurethane piece is then soaked in concentrated hydrochloric acid for a sufficient amount of time to allow the acid to dissolve the polyurethane foam. The resulting open cell surface silicone foam is then washed in deionized water. The resulting removal of the polyurethane foam produces a 20 cm×20 cm×5 mm silicone foam sheet having a first 20 cm×20 cm surface comprising an open cell structure and a second 20 cm×20 cm surface comprising an open-cell surface.

A sample from the sheet of silicone foam material can be characterized by microCT analysis and/or scanning electron microscopy (SEM).

### Example 3

### A Method of Making a Breast Implant Comprising an Open Cell Surface Foam Material

This example illustrates how to make a biocompatible implantable device comprising an open cell surface foam material as disclosed in the present specification.

Sheets of silicone foam material are obtained as described in Example 1. Alternatively, sheets of silicone foam material are obtained as described in Example 2.

To attach a silicone foam material to a biocompatible implantable device, a first silicone foam sheet is coated with a thin layer of silicone on its closed cell surface and then placed in the bottom cavity of a mold, adhesive side up. A breast implant is then placed on top of the foam surface coated with the adhesive. A second silicone foam material sheet is then coated with a thin layer of silicone on its closed cell surface and applied to the uncovered surface of the implant. The top piece of the mold cavity is then fixed in place pressing the two foam sheets together creating a uniform interface. The silicone adhesive is allowed to cure by placing the covered device into an oven and heated at a temperature of 126° C. for 75 minutes. After curing, excess foam material is trimmed off creating a uniform seam around the breast implant. This process results in a breast implant comprising a foam material as disclosed in the present specification. See, e.g., FIG. 2.

Alternatively, the foam material can be laminated onto a breast implant while the device is still on a mandrel. In this process, a first porous material sheet is coated with a thin layer of silicone and then draped over the device on the mandrel in such a way that there are no wrinkles on the surface. After curing the silicone adhesive, as described above, another coating of silicone is applied to the uncovered surface of the biocompatible implantable device and a second porous material is stretched up to cover the back of the device. After curing the silicone adhesive, as described above, the biocompatible implantable device is then taken off the mandrel and the excess porous material is trimmed to create a uniform seam around the device. This process results in a biocompatible implantable device comprising a porous material as disclosed in the present specification. See, e.g., FIG. 2.

### Example 4

### A Method of Making an Open Cell Surface Foam Material Shell

This example illustrates how to make an open cell surface foam material shell as disclosed in the present specification.

To prepare a mold assembly, a multi-component mold assembly is assembled such that an internal space is created in the shape of a breast implant shell. In its design, the component part or parts of the mold assemble forming the convex surface of the shell has a textured surface comprising an array of interconnected void space. This textured surface component part or parts can be produced by reversibly affixing a polyurethane foam to the component part.

A foaming solution is prepared as described in Example 1.

The silicone foaming solution is then added to the mold assembly in a manner that allows penetration or integration of the solution into the void space of the polyurethane foam. The silicone foaming solution is then treated by allowing the solution to stand at ambient temperature until the foaming solution cured.

The mold assembly is then dismantled to remove the silicone foam material from the assembly. During this dismantling procedure, the polyurethane foam physically removes a surface portion of the cured silicone foam material shell at or near the site of penetration or integration into the void spaces to create a silicone foam material having an open cell surface. The resulting removal of the polyurethane foam produces a silicone foam shell having the shape of a breast implant where the concave portion comprising a closed-cell surface and the convex portion comprising an open-cell surface. See, e.g., FIG. 3.

Alternatively, the mold assembly may be dismantled in such a manner that leaves the polyurethane foam in contact with the silicone foam. This silicone-polyurethane piece is then soaked in concentrated hydrochloric acid for a sufficient amount of time to allow the acid to dissolve the polyurethane foam. The resulting open cell surface silicone foam is then washed in deionized water. The resulting removal of the polyurethane foam produces a silicone foam shell having the shape of a breast implant where the concave portion comprising a closed-cell surface and the convex portion comprising an open-cell surface. See, e.g., FIG. 3.

A sample from the sheet of silicone foam material can be characterized by microCT analysis and/or scanning electron microscopy (SEM).

### Example 5

### A Method of Making an Open Cell Surface Foam Material Shell

This example illustrates how to make an open cell surface foam material shell as disclosed in the present specification.

To prepare a mold assembly, a multi-component mold assembly is assembled such that an internal space is created in the shape of a breast implant shell. In its design, the component part or parts of the mold assemble forming the convex surface of the shell has a textured surface comprising an array of interconnected void space. In addition, the component part or parts of the mold assemble forming the concave surface of the shell has a textured surface comprising an array of interconnected void space. These textured surface component parts can be produced by reversibly affixing a polyurethane foam to the component part.

A foaming solution is prepared as described in Example 1.

The silicone foaming solution is then added to the mold assembly in a manner that allows penetration or integration of the solution into the void space of the polyurethane foam of the component parts comprising the convex and concave surfaces of the shell. The silicone foaming solution is then treated by allowing the solution to stand at ambient temperature until the foaming solution cured.

The mold assembly is then dismantled to remove the silicone foam material from the assembly. During this dismantling procedure, the polyurethane foam from both the convex and concave surfaces physically removes a surface portion of the cured silicone foam material shell at or near the site of penetration or integration into the void spaces to create a silicone foam material having an open cell surface. The resulting removal of the polyurethane foam produces a silicone foam shell having the shape of a breast implant where the concave portion comprising a closed-cell surface and the convex portion comprising an open-cell surface. See, e.g., FIG. 3.

Alternatively, the mold assembly may be dismantled in such a manner that leaves both the polyurethane foam from the convex and concave surfaces in contact with the silicone foam. This silicone-polyurethane piece is then soaked in concentrated hydrochloric acid for a sufficient amount of time to allow the acid to dissolve the polyurethane foam. The resulting open cell surface silicone foam is then washed in deionized water. The resulting removal of the polyurethane foam produces a silicone foam shell having the shape of a breast implant where the concave portion comprising a closed-cell surface and the convex portion comprising an open-cell surface. See, e.g., FIG. 3.

A sample from the sheet of silicone foam material can be characterized by microCT analysis and/or scanning electron microscopy (SEM).

### Example 6

### A Method of Making a Biocompatible Implantable Device Comprising a Porous Material

This example illustrates how to make a biocompatible implantable device comprising a porous material disclosed in the present specification.

A silicone foam shell is obtained as described in Example 4. Alternatively, a silicone foam material is obtained as described in Example 5.

To attach the silicone foam shell to a breast implant, the surface of the implant is coated with a thin layer of silicone. The concave portion of the foam shell is then placed over the adhesive coated device in a manner that ensures no wrinkles in the foam shell. The silicone adhesive is allowed to cure by placing the covered implant into an oven and heating at a temperature of 126° C. for 75 minutes. After curing, excess foam material is trimmed off. This process results in a breast implant comprising a foam material having an open cell surface as disclosed in the present specification. See, e.g., FIG. 4.

In closing, it is to be understood that although aspects of the present specification have been described with reference to the various embodiments, one skilled in the art will readily appreciate that the specific examples disclosed are only illustrative of the principles of the subject matter disclosed in the present specification. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

All patents, patent publications, and other publications referenced and identified in the present specification are individually and expressly incorporated herein by reference in their entirety for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed:

1. A breast implant shell comprising:
a fillable envelope including an open-cell foam element defining an anterior surface of the envelope, the element produced by the steps of:
a. preparing a foaming solution, the foaming solution comprising an elastomer base and a curing agent;
b. adding the foaming solution to a mold assembly having a textured surface component having a surface porosity of at least about 40%, a textured surface of the textured surface component being defined by an interconnected array of void space, wherein the foaming solution is added to the mold assembly in a manner sufficient to allow penetration of the foaming solution into the interconnected array of void space;
c. curing the foaming solution to produce an elastomeric foam material on the textured surface component; and
d. removing the textured surface component from the elastomeric foam material to obtain a foam material having an open-cell surface.

2. The implant of claim 1, wherein the textured surface component is a foam, a felt, a mesh, a bundle of fibers, a sintered surface, or a screen.

3. The implant of claim 2, wherein the textured surface component is a foam.

4. The implant of claim 2, wherein the foam is a polyurethane-based foam, a melamine foam, a polyolefin foam, a silicone foam.

5. The implant of claim 1 wherein the textured surface component is a polyurethane based foam.

6. The implant of claim 1, wherein the surface porosity of the textured surface component is between about 40% and about 90%.

7. The implant of claim 1 wherein the step of removing includes pulling the elastomeric foam material apart from the textured surface component.

8. The implant of claim 7 wherein the step of removing further comprising contacting the elastomeric foam material with an agent capable of dissolving any of the textured foam material remaining attached to the elastomeric foam material.

9. The implant of claim 5 wherein the step of removing comprises pulling the elastomeric foam material apart from the textured surface component and further comprises contacting the elastomeric foam material with an agent capable of dissolving any of the textured foam material remaining attached to the elastomeric foam material after the step of pulling.

10. A method of making a textured material useful as a component of a fillable breast implant shell, the method comprising the steps of:
a. preparing a foaming solution, the foaming solution comprising an elastomer base and a curing agent;
b. adding the foaming solution to a mold assembly having a textured surface component having a surface porosity of at least about 40%, a textured surface of the textured surface component being defined by an interconnected array of void space, wherein the foaming solution is added to the mold assembly in a manner sufficient to allow penetration of the foaming solution into the interconnected array of void space;
c. curing the foaming solution to produce an elastomeric foam material on the textured surface component; and
d. removing the textured surface component from the elastomeric foam material to obtain a foam material having an open-cell surface.

11. The method of claim 10, wherein the textured surface component is a foam, a felt, a mesh, a bundle of fibers, a sintered surface, or a screen.

12. The method of claim 11, wherein the textured surface component is an elastomer-based foam, a ceramic-based foam, or a metal-composite-based foam.

13. The method of claim 11, wherein the foam is a polyurethane-based foam, a melamine foam, a polyolefin foam, a silicone foam.

14. The method of claim 11, wherein the textured surface component felt is a polypropylene-based felt or a cellulose-based felt.

15. The method of claim 10, wherein the textured surface component comprises a separate component that is reversibly attached to another component part of the mold assembly.

16. A biocompatible open-cell porous material useful as a component of an implantable device, the material made by a method comprising the steps of:
a. preparing a foaming solution, the foaming solution comprising an elastomer base and a curing agent;
b. adding the foaming solution to a mold assembly, the mold assemble comprising a textured surface component having an interconnected array of void space, wherein the foaming solution is added to the mold assembly in a manner sufficient to allow penetration of the foaming solution into the interconnected array of void space;
c. treating the foaming solution to produce a foam material; and
d. removing the mold assembly, wherein removal of the textured surface component produces a foam material having an open-cell surface.

17. The material of claim 16, wherein the foam material comprises silicone and the textured surface component comprises a polyurethane-based foam having a surface porosity of at least 40%.
